(19) United States
(12) Patent Application Publication
(10) Pub. No.: US 2022/0233209 A1
(43) Pub. Date: Jul. 28, 2022

(54) COLONIC TREATMENT METHODS AND APPARATUS

(71) Applicants: Thomas Julius BORODY, Five Dock (AU); Antony MILIS, Sydney (AU)

(72) Inventors: Thomas Julius BORODY, Five Dock (AU); Antony MILIS, Sydney (AU)

(21) Appl. No.: 17/615,253

(22) PCT Filed: Jul. 30, 2020
(86) PCT No.: PCT/AU2020/050774

§ 371 Date: Nov. 30, 2021

Related U.S. Application Data
(60) Provisional application No. 62/882,412, filed on Aug. 2, 2019.

(51) Int. Cl. A61B 17/22; A61B 1/31

(52) U.S. Cl. CPC A61B 17/2202; A61B 1/31; A61B 2017/00296; A61B 2017/22028; A61B 2017/22021

(57) ABSTRACT

Provided are products of manufacture and methods for the removal and/or destruction of a biofilm in situ, e.g., a gastro-luminal biofilm, and for the treatment or amelioration of biofilm-associated diseases, infections and conditions, including and GI luminal infections. Provided are devices and apparatus, and methods for using them, for the removal, disruption and/or destruction of a biofilm in situ, e.g., a gastro-luminal biofilm. In alternative embodiments, provided are devices and apparatus, and methods, for enhancing biofilm dissolving or disrupting agents, or for administering biofilm dissolving or disrupting agents, where in alternative embodiments the biofilm comprises a gastro-luminal ‘unstirred layer’, adherent layer or gastro-luminal mucus layer; or the biofilm comprises a matrix or a DNA-containing layer, or alternatively the biofilm comprises a polysaccharide gastro-luminal peripheral layer.

### CROSS-REFERENCE TO RELATED APPLICATION

The present application claims priority to US 62/882,412, filed on Aug. 2, 2019, the entire disclosure of which is incorporated herein by cross-reference.

### FIELD

This invention generally relates to gastroenterology and colonic microbiome biology and engraftment. Provided are products of manufacture and methods for the removal and/or destruction of a biofilm in situ, e.g., a gastro-luminal biofilm, and for the treatment or amelioration of biofilm-associated diseases, infections and conditions, including and GI luminal infections, e.g., in preparation for fecal microbiota transplantation (FMT). Provided are devices and apparatus, and methods for using them, for the removal, disruption and/or destruction of a biofilm in situ, e.g., a gastro-luminal biofilm. In alternative embodiments, provided are devices and apparatus, and methods, for enhancing biofilm dissolving or disrupting agents, or for administering biofilm dissolving or disrupting agents, where in alternative embodiments the biofilm comprises a gastro-luminal ‘unstirred layer’, adherent layer or gastro-luminal mucus layer; or the biofilm comprises a matrix or a DNA-containing layer, or alternatively the biofilm comprises a polysaccharide gastro-luminal peripheral layer.

### BACKGROUND

Recent advances in microbiology and microbiomics, and in particular those of various microbiomes in animals, have shown that the root cause of many acute but also chronic conditions including autoimmune and neurological diseases, are based on a dysbiotic gut microbiome where an occult infection or multiple infections resides in the stool, and particularly in the biofilm, which makes up the unstirred or adherent layer on the edges of the gastrointestinal lumen, the barrier between the gut microbiome and the tissue mucosa.

For example, in Inflammatory Bowel Disease (IBD) this unstirred or adherent layer of biofilm comprises Bacteroides fragilis, which can be stained using fluorescent in situ hybridization (FISH), and B. fragilis makes up a considerable proportion of the microbiome film stain. The causal component, i.e., the infective (pathogenic) agent of the biofilm, is generally difficult to identify as only small numbers of pathogens are needed to initiate inflammation; often their genus of the infective agent of the biofilm is yet to be named. In Ulcerative Colitis (UC) the infective agent of the biofilm is mostly Fusobacteria, which is difficult to detect and may be the cause of UC mucosal inflammation.

The largest microbiome in humans and many animals resides in the gastrointestinal (GI) tract. It may become infected and dysbiotic by various infective (pathogenic) agents, for example, bacteria, resulting in various diseases based on an abnormal microbiome. Such disorders include Clostridioides difficile infection (CDI), which is the best-studied example. Numerous other infectious and toxin-producing states such as those mediating UC, Crohn's disease (CD), constipation and food sensitivities have been detected based on their responsiveness to antibiotic treatments, but specific causative agents have yet to be definitively identified.

In GI-related diseases and conditions such as UC, antibiotics can induce a temporary improvement of symptoms by suppression of infective (pathogenic) agents in the gut's biofilm; however, the disease or condition eventually returns after the cessation of the antibiotic treatment because the infection continues to reside in the biofilm where the infective agents survive the antibiotic's effects. In a similar fashion this is seen with CDI and Helicobacter pylori infections.

Attempts to remove the infected biofilm, which would allow replacement with a new microbiome composition that does not contain a pathogen or an infective (pathogenic) agent, to date have not been effective. Thus, Faecal Microbiota Transplantation (FMT), which entails infusion into the lumen of the gut of an infected or afflicted individual an uninfected or non-disease-causing donor gut microbiome with the goal of replacing the pathogenic microbiome, has generally to date not been sufficiently effective to achieve its desired results, which can include eradication of a gut's pathogens—except in CDI where the biofilm is quite fragile. A major problem with use of FMT has been identified as a difficulty in efficiently removing the existing pathogenic biofilm to implant the new donor microbiome

Apart from CDI, a universal problem is that after one or more antibiotic rounds of treatment, and even after repeated FMT, a return or relapse of symptoms occurs. This return or relapse appears to be due to the persisting, infected (pathogenic) surface mucosal ‘unstirred layer’ of biofilm. This biofilm layer can be of various thickness, and can comprise various polysaccharides, DNA, mucus and resident bacteria. The ‘unstirred layer’ of biofilm can protect pathogenic bacteria such that even high doses and concentrations of multiple antibiotics cannot generally cure these ‘intra-biofilm’ infections. The protection of the biofilm-comprising pathogenic bacteria from the antibiotics and the individual's immune system permits the infective condition to persist and be difficult to impossible to cure. Therefore, it is necessary that an anti-biofilm approach be developed to address the root cause of such dysbioses.

Some diseases that have been shown to have a dysbiotic gut microbiome include Ulcerative Colitis (UC), Crohn's Disease (CD), Parkinson's Disease (PD), Multiple Sclerosis (MS), epilepsy, Autism Spectrum Disease (ASD), ITP, anorexia nervosa, rheumatoid arthritis and alopecia totalis, to name just a few. The abovementioned and other conditions are currently being treated with medications that have poor efficacy against the (pathogenic) biofilm, or there is currently no medication available to provide adequate relief to these patients.

In addition to the above illnesses, pathogen-infected biofilm may play a role in other conditions in the human body including but not limited to, resistant Helicobacter pylori infection of the stomach, sinusitis, lung infections (for example, as cystic fibrosis, bronchiectasis and asthma), bladder infections (for example, interstitial cystitis).

Therefore, it is of vital clinical importance to introduce therapies which aim to remove the pathogenic biofilm to offer a new and effective treatment to patients where biofilm is the limiting factor in the resolution of their medical condition. This may require complete replacement of the luminal microbiota and biofilm with an uninfected (non-pathogenic) donor faecal flora which will form the new, uninfected (non-pathogenic) biofilm and result in luminal microbiome that promotes a healthy condition.

Ultrasound cleaning devices have relied mostly on longitudinal waves emanating from an ultrasound tip at the distal end of an apparatus to deliver the ultrasound to the target tissue under a flowing fluid stream which acts as an acoustic coupler as in dentistry. Due to the small size of such probes the area treated is usually small and the management of the flowing liquid coupler in an internal body cavity is challenging in a clinical setting of the gut lumen. This drawback makes this type of various ultrasound devices inefficient when trying to treat larger tissue surface areas, for example, the large intestine wall. Apart from ultrasonic damage to biofilm, jets of liquid delivered to the lumen of the bowel have been used to enhance, but not effectively remove, biofilm.

### SUMMARY

There is provided herein products of manufacture for debriding or disrupting a biofilm in situ, comprising an endoscope having an outer body or sheath and an inner lumen, wherein the endoscope comprises one or a plurality of ultrasound emitters, and/or one or a plurality of ultrasound ring-shaped transducers,

and the one or a plurality of ultrasound emitters, or the one or a plurality of ring-shaped ultrasound transducers, each generate an ultrasound wave that can travel perpendicularly and/or radially away from the longitudinal axis of the endoscope,

wherein optionally the one or a plurality of ultrasound emitters are flexible and are wrapped around the outer body of the endoscope, or the one or a plurality of ultrasound emitters are placed or positioned on the outer body of the endoscope, and optionally the one or a plurality of ultrasound emitters are flat against the outer body or sheath or do not substantially protrude from or minimally protrudes from the outer body or sheath,

and optionally the one or a plurality of ultrasound emitters are placed or positioned within or attached to the inside of the outer body or sheath, and the outer body or sheath substantially comprises a material which does not attenuate or alter the frequency of an ultrasound wave emitted by the one or a plurality of ultrasound emitters,

and optionally the one or a plurality of ultrasound emitters comprise one or a plurality of disk-shaped ultrasound emitters at regular intervals horizontally and longitudinally along the endoscope body, optionally placed as illustrated in FIG. 8,

and optionally the one or a plurality of ultrasound emitters, or the one or the plurality of ring-shaped ultrasound transducers, are placed or positioned along the distal half of the length of the endoscope, or are placed or positioned along the along the distal third or quarter of the length of the endoscope,

and optionally the one or a plurality of ultrasound emitters, or the one or the plurality of ring-shaped ultrasound transducers, transmit ultrasound energy in a continuous or pulsed mode.

In alternative embodiments of products of manufacture as provided herein:

the products of manufacture further comprise an electrode or electric cable running thorough the inner lumen, and the electrode cable is operatively connected to the one or a plurality of ultrasound emitters, or the one or plurality of ring-shaped ultrasound transducers, to power the one or a plurality of ultrasound emitters or the one or plurality of ring-shaped ultrasound transducers;

the products of manufacture further comprise one or a plurality of temperature sensors,

wherein optionally the one or a plurality of temperature sensors are spaced along the length of the endoscope,

and optionally the one or a plurality of temperature sensors are operatively connected to a display or a control panel that displays a temperature reading by the one or a plurality of temperature sensors to an operator,

and optionally the one or a plurality of temperature sensors are operatively connected to a computer capable of monitoring the temperature and turning off the power to the one or a plurality of ultrasound emitters when the temperature reaches a predetermined temperature setting;

the one or plurality of ring-shaped transducers are placed at regular intervals inside the entire length, or along a section of, the endoscope's sheath or outer body,

wherein optionally the ring-shaped transducers are placed every 3 to 10 or 20 cm, or every 10 to 30 cm, along the length of and inside the endoscope's sheath or outer body, and optionally the ring-shaped transducers are placed or positioned along the distal half of the length of the endoscope, or are placed or positioned along the along the distal third or quarter of the length of the endoscope;

the products of manufacture further comprise a wide beam ultrasound emitter array,

wherein optionally the wide beam ultrasound emitter array is a built-in component of the endoscope or is an attachment to the endoscope, and optionally the wide beam ultrasound emitter array is a removable attachment to the endoscope,

and optionally the wide beam ultrasound emitter array transmits ultrasound waves perpendicularly and/or radially away from the longitudinal axis of the endoscope,

and optionally the wide beam ultrasound emitter array comprises a device as set forth in FIG. 5 or FIG. 6,

and optionally the wide beam ultrasound emitter array is positioned no closer to between about 5 cm to 20 cm from the distal end or tip of the endoscope,

and optionally the electrode or electric cable running thorough the inner lumen is operatively connected to the wide beam ultrasound emitter array and powers the wide beam ultrasound emitter array,

and optionally the wide beam ultrasound emitter array transmits ultrasound energy in a continuous or pulsed mode,

and optionally the wide beam ultrasound emitter array has a curvilinear shape,

and optionally the wide beam ultrasound emitter array comprises a lifting mechanism which can lift the wide beam ultrasound emitter array to between 1 to 90 degrees from the longitudinal axis of the endoscope, and optionally the lifting mechanism is operatively connected to a control mechanism capable of activating the lifting mechanism to lift the wide beam ultrasound emitter array to between 1 to 90 degrees from the longitudinal axis of the endoscope, or to close the wide beam ultrasound emitter array back against the body of the endoscope;

the products of manufacture further comprise a plurality of spacing rings projecting out from the body of the product of manufacture at regular intervals to prevent the product of manufacture from resting against a tissue when the product of manufacture is inserted into the body, optionally inserted into a colon,

and optionally the plurality of spacing rings project out from the body of the product of manufacture between about 2 to 20 cm,

and optionally the plurality of spacing rings are spaced between about 3 to 30 cm along the length of the product of manufacture,

and optionally the plurality of spacing rings comprise flexible spacing rings;

the products of manufacture further comprise an attachment that is secured to the distal end of the product of manufacture, wherein the attachment comprises an array of radial ultrasound emitters, optionally ring ultrasound transducers, situated or placed intermittently along the length of the attachment, and the attachment is operably connected to the electrode or electric cable running thorough the inner lumen of the product of manufacture to power the radial ultrasound emitters,

and the optionally the attachment further comprises a plurality of spacing rings situated or placed intermittently along the length of the attachment,

and optionally the attachment has a rounded tip or end, and optionally the rounded tip or end comprises a plurality of bristles or equivalent protrusions,

and optionally the attachment is configured as set forth in FIG. 9;

the products of manufacture further comprise a vibrating motor operatively connected to an external control unit via a cable, and the vibrating motor is operatively connected to a waveguide with a rounded tip or end that can be extended past or outside of the distal end of the product of manufacture, and the vibrating motor when activated causes the rounded tip or end to vibrate in an oscillating motion,

and optionally the rounded tip or end comprises a plurality of bristles or equivalent protrusions,

and optionally the vibrating motor, waveguide and rounded tip or end are configured as set forth in FIG. 12a or FIG. 12b;

the products of manufacture further comprise a microscope array, optionally a confocal microscope array, built-into the end or tip of the product of manufacture;

the products of manufacture further comprise: (a) a plurality of liquid spray holes; (b) a plurality of aspiration openings; or (c) a combination of (a) and (b),

and the plurality of aspiration openings are operatively connected to a plurality of tubes to allow aspiration of fluids or liquids from a tissue space surrounding the product of manufacture when the product of manufacture is inserted in a body space in situ,

and the plurality of liquid spray holes are operatively connected to a plurality of tubes to allow spraying or ejection of fluids or liquids under pressure out from the product of manufacture into a tissue space surrounding the product of manufacture when the product of manufacture is inserted in a body space in situ, and optionally the plurality of liquid spray holes are extended, angled or pointed back away from the distal end of the product of manufacture, optionally configured to allow a liquid or a fluid sprayed from the plurality of liquid spray holes to cleanse the product of manufacture,

wherein optionally plurality of aspiration openings have a larger diameter than the plurality of liquid spray holes,

and optionally the plurality of aspiration openings and/or the plurality of liquid spray holes are configured as set forth in FIG. 13 or FIG. 14,

and the plurality of aspiration openings and/or the plurality of liquid spray holes are situated in the distal half, third or quarter end of the product of manufacture;

the products of manufacture further comprise an overtube fitted along the outer circumference of the product of manufacture,

wherein optionally the overtube comprises plurality of liquid spray holes operatively connected to a plurality of tubes to allow spraying or ejection of fluids or liquids under pressure out from the product of manufacture into a tissue space surrounding the product of manufacture when the product of manufacture is inserted in a body space in situ, and optionally the plurality of liquid spray holes are extended, angled or pointed back away from the distal end of the product of manufacture, optionally configured to allow a liquid or a fluid sprayed from the plurality of liquid spray holes to cleanse the product of manufacture,

and optionally the overtube comprises plurality of aspiration openings operatively connected to a plurality of tubes to allow aspiration of fluids or liquids from a tissue space surrounding the product of manufacture when the product of manufacture is inserted in a body space in situ,

and optionally the overtube comprises a one or a plurality channels having lumens capable of having inserted therein a tube or an instrument, and optionally an instrument inserted into one or in a plurality channels is capable of delivering and inflating a balloon into a body space in situ, and optionally an instrument inserted into one or in a plurality channels is capable of delivering a therapeutic solution or formulation, and optionally the therapeutic solution or formulation comprises a biofilm dissolving or disrupting agent, a soap, an antibiotic or a fecal microbiota transplantation formulation,

and optionally the plurality of aspiration openings and/or the plurality of liquid spray holes are configured as set forth in FIG. 15a or FIG. 15b;

the products of manufacture are configured or manufactured as a product of manufacture, device or endoscope as set forth in FIG. 1, FIG. 2, FIG. 3, FIG. 4, FIG. 7, FIG. 9, FIG. 10A, FIG. 10B, FIG. 11A, FIG. 11B, FIG. 12A, FIG. 12B, FIG. 13, FIG. 14, FIG. 15A or FIG. 15B.

In alternative embodiments, provided are methods for debriding or disrupting a biofilm in situ, comprising a product of manufacture as provided herein.

In alternative embodiments, provided are Uses of a product of manufacture as provided herein, or a kit as provided herein, for debriding or disrupting a biofilm in situ.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

Forms of the invention include the following:
1. A product of manufacture for debriding or disrupting a biofilm in situ, comprising an endoscope having an outer body or sheath and an inner lumen, wherein the endoscope comprises one or a plurality of ultrasound emitters, and/or one or a plurality of ultrasound ring-shaped transducers,

and the one or a plurality of ultrasound emitters, or the one or a plurality of ring-shaped ultrasound transducers, each generate an ultrasound wave that can travel perpendicularly and/or radially away from the longitudinal axis of the endoscope.
2. The product of manufacture of form 1,

wherein the one or a plurality of ultrasound emitters are flexible and are wrapped around the outer body of the endoscope, or the one or a plurality of ultrasound emitters are placed or positioned on the outer body of the endoscope, and optionally the one or a plurality of ultrasound emitters are flat against the outer body or sheath or do not substantially protrude from or minimally protrudes from the outer body or sheath.
3. The product of manufacture of form 1 or 2,

wherein the one or a plurality of ultrasound emitters are placed or positioned within or attached to the inside of the outer body or sheath, and the outer body or sheath substantially comprises a material which does not attenuate or alter the frequency of an ultrasound wave emitted by the one or a plurality of ultrasound emitters.
4. The product of manufacture of any one of the preceding forms,

wherein the one or a plurality of ultrasound emitters comprise one or a plurality of disk-shaped ultrasound emitters at regular intervals horizontally and longitudinally along the endoscope body, optionally placed as illustrated in FIG. 8.
5. The product of manufacture of any one of the preceding forms,

wherein the one or a plurality of ultrasound emitters, or the one or the plurality of ring-shaped ultrasound transducers, are placed or positioned along the distal half of the length of the endoscope, or are placed or positioned along the along the distal third or quarter of the length of the endoscope.
6. The product of manufacture of any one of the preceding forms,

wherein the one or a plurality of ultrasound emitters, or the one or the plurality of ring-shaped ultrasound transducers, transmit ultrasound energy in a continuous or pulsed mode.
7. The product of manufacture of any one of the preceding forms, further comprising an electrode or electric cable running thorough the inner lumen, and the electrode cable is operatively connected to the one or a plurality of ultrasound emitters, or the one or plurality of ring-shaped ultrasound transducers, to power the one or a plurality of ultrasound emitters or the one or plurality of ring-shaped ultrasound transducers.
8. The product of manufacture of any one of the preceding forms, further comprising one or a plurality of temperature sensors,

wherein optionally the one or a plurality of temperature sensors are spaced along the length of the endoscope,

and optionally the one or a plurality of temperature sensors are operatively connected to a display or a control panel that displays a temperature reading by the one or a plurality of temperature sensors to an operator,

and optionally the one or a plurality of temperature sensors are operatively connected to a computer capable of monitoring the temperature and turning off the power to the one or a plurality of ultrasound emitters when the temperature reaches a predetermined temperature setting.
9. The product of manufacture of any of the preceding forms, wherein the one or plurality of ring-shaped transducers are placed at regular intervals inside the entire length, or along a section of, the endoscope' s sheath or outer body,

wherein optionally the ring-shaped transducers are placed every 3 to 10 or 20 cm, or every 10 to 30 cm, along the length of and inside the endoscope' s sheath or outer body,

and optionally the ring-shaped transducers are placed or positioned along the distal half of the length of the endoscope, or are placed or positioned along the along the distal third or quarter of the length of the endoscope.
10. The product of manufacture of any of the preceding forms, further comprising a wide beam ultrasound emitter array,

wherein optionally the wide beam ultrasound emitter array is a built-in component of the endoscope or is an attachment to the endoscope, and optionally the wide beam ultrasound emitter array is a removable attachment to the endoscope,

and optionally the wide beam ultrasound emitter array transmits ultrasound waves perpendicularly and/or radially away from the longitudinal axis of the endoscope,

and optionally the wide beam ultrasound emitter array comprises a device as set forth in FIG. 5 or FIG. 6,

and optionally the wide beam ultrasound emitter array is positioned no closer to between about 5 cm to 20 cm from the distal end or tip of the endoscope,

and optionally the electrode or electric cable running thorough the inner lumen is operatively connected to the wide beam ultrasound emitter array and powers the wide beam ultrasound emitter array,

and optionally the wide beam ultrasound emitter array transmits ultrasound energy in a continuous or pulsed mode,

and optionally the wide beam ultrasound emitter array has a curvilinear shape,

and optionally the wide beam ultrasound emitter array comprises a lifting mechanism which can lift the wide beam ultrasound emitter array to between 1 to 90 degrees from the longitudinal axis of the endoscope, and optionally the lifting mechanism is operatively connected to a control mechanism capable of activating the lifting mechanism to lift the wide beam ultrasound emitter array to between 1 to 90 degrees from the longitudinal axis of the endoscope, or to close the wide beam ultrasound emitter array back against the body of the endoscope.
11. The product of manufacture of any of the preceding forms, wherein the product of manufacture further comprises a plurality of spacing rings projecting out from the body of the product of manufacture at regular intervals to prevent the product of manufacture from resting against a tissue when the product of manufacture is inserted into the body, optionally inserted into a colon,

and optionally the plurality of spacing rings project out from the body of the product of manufacture between about 2 to 20 cm,

and optionally the plurality of spacing rings are spaced between about 3 to 30 cm along the length of the product of manufacture,

and optionally the plurality of spacing rings comprise flexible spacing rings.
12. The product of manufacture of any of the preceding claims, wherein the product of manufacture further comprising an attachment that is secured to the distal end of the product of manufacture, wherein the attachment comprises an array of radial ultrasound emitters, optionally ring ultrasound transducers, situated or placed intermittently along the length of the attachment, and the attachment is operably connected to the electrode or electric cable running thorough the inner lumen of the product of manufacture to power the radial ultrasound emitters,

and the optionally the attachment further comprises a plurality of spacing rings situated or placed intermittently along the length of the attachment,

and optionally the attachment has a rounded tip or end, and optionally the rounded tip or end comprises a plurality of bristles or equivalent protrusions,

and optionally the attachment is configured as set forth in FIG. 9.
13. The product of manufacture of any of the preceding forms, wherein the product of manufacture further comprises a vibrating motor operatively connected to an external control unit via a cable, and the vibrating motor is operatively connected to a waveguide with a rounded tip or end that can be extended past or outside of the distal end of the product of manufacture, and the vibrating motor when activated causes the rounded tip or end to vibrate in an oscillating motion,

and optionally the rounded tip or end comprises a plurality of bristles or equivalent protrusions,

and optionally the vibrating motor, waveguide and rounded tip or end are configured as set forth in FIG. 12a or FIG. 12b.
14. The product of manufacture of any of the preceding forms, wherein the product of manufacture further comprises a microscope array, optionally a confocal microscope array, built-into the end or tip of the product of manufacture.
15. The product of manufacture of any of the preceding forms, wherein the product of manufacture further comprises: (a) a plurality of liquid spray holes; (b) a plurality of aspiration openings; or (c) a combination of (a) and (b),

and the plurality of aspiration openings are operatively connected to a plurality of tubes to allow aspiration of fluids or liquids from a tissue space surrounding the product of manufacture when the product of manufacture is inserted in a body space in situ,

and the plurality of liquid spray holes are operatively connected to a plurality of tubes to allow spraying or ejection of fluids or liquids under pressure out from the product of manufacture into a tissue space surrounding the product of manufacture when the product of manufacture is inserted in a body space in situ, and optionally the plurality of liquid spray holes are extended, angled or pointed back away from the distal end of the product of manufacture, optionally configured to allow a liquid or a fluid sprayed from the plurality of liquid spray holes to cleanse the product of manufacture,

wherein optionally plurality of aspiration openings have a larger diameter than the plurality of liquid spray holes,

and optionally the plurality of aspiration openings and/or the plurality of liquid spray holes are configured as set forth in FIG. 13 or FIG. 14,

and the plurality of aspiration openings and/or the plurality of liquid spray holes are situated in the distal half, third or quarter end of the product of manufacture.
16. The product of manufacture of any of the preceding forms, further comprising an overtube fitted along the outer circumference of the product of manufacture,

wherein optionally the overtube comprises plurality of liquid spray holes operatively connected to a plurality of tubes to allow spraying or ejection of fluids or liquids under pressure out from the product of manufacture into a tissue space surrounding the product of manufacture when the product of manufacture is inserted in a body space in situ, and optionally the plurality of liquid spray holes are extended, angled or pointed back away from the distal end of the product of manufacture, optionally configured to allow a liquid or a fluid sprayed from the plurality of liquid spray holes to cleanse the product of manufacture,

and optionally the overtube comprises plurality of aspiration openings operatively connected to a plurality of tubes to allow aspiration of fluids or liquids from a tissue space surrounding the product of manufacture when the product of manufacture is inserted in a body space in situ,

and optionally the overtube comprises a one or a plurality channels having lumens capable of having inserted therein a tube or an instrument, and optionally an instrument inserted into one or in a plurality channels is capable of delivering and inflating a balloon into a body space in situ, and optionally an instrument inserted into one or in a plurality channels is capable of delivering a therapeutic solution or formulation, and optionally the therapeutic solution or formulation comprises a biofilm dissolving or disrupting agent, a soap, an antibiotic or a fecal microbiota transplantation formulation,

and optionally the plurality of aspiration openings and/or the plurality of liquid spray holes are configured as set forth in FIG. 15a or FIG. 15b.
17. The product of manufacture of any of the preceding forms, wherein the product of manufacture is configured or manufactured as a product of manufacture, device or endoscope as set forth in FIG. 1, FIG. 2, FIG. 3, FIG. 4, FIG. 7, FIG. 9, FIG. 10A, FIG. 10B, FIG. 11A, FIG. 11B, FIG. 12A, FIG. 12B, FIG. 13, FIG. 14, FIG. 15A or FIG. 15B.
18. A method for debriding or disrupting a biofilm in situ, comprising use of a product of manufacture of any of the preceding forms.
19. Use of a product of manufacture of any of the preceding forms, or a kit of any of the preceding claims, for debriding or disrupting a biofilm in situ.
20. A product of manufacture for debriding or disrupting a biofilm in situ, comprising an endoscope having an outer body or sheath and an inner lumen, wherein the endoscope comprises one or a plurality of ultrasound emitters, and/or one or a plurality of ultrasound ring-shaped transducers,

and the one or a plurality of ultrasound emitters, or the one or a plurality of ring-shaped ultrasound transducers, each generate an ultrasound wave that can travel perpendicularly and/or radially away from the longitudinal axis of the endoscope,

wherein optionally the one or a plurality of ultrasound emitters are flexible and are wrapped around the outer body of the endoscope, or the one or a plurality of ultrasound emitters are placed or positioned on the outer body of the endoscope, and optionally the one or a plurality of ultrasound emitters are flat against the outer body or sheath or do not substantially protrude from or minimally protrudes from the outer body or sheath,

and optionally the one or a plurality of ultrasound emitters are placed or positioned within or attached to the inside of the outer body or sheath, and the outer body or sheath substantially comprises a material which does not attenuate or alter the frequency of an ultrasound wave emitted by the one or a plurality of ultrasound emitters,

and optionally the one or a plurality of ultrasound emitters comprise one or a plurality of disk-shaped ultrasound emitters at regular intervals horizontally and longitudinally along the endoscope body, optionally placed as illustrated in FIG. 8,

and optionally the one or a plurality of ultrasound emitters, or the one or the plurality of ring-shaped ultrasound transducers, are placed or positioned along the distal half of the length of the endoscope, or are placed or positioned along the along the distal third or quarter of the length of the endoscope,

and optionally the one or a plurality of ultrasound emitters, or the one or the plurality of ring-shaped ultrasound transducers, transmit ultrasound energy in a continuous or pulsed mode.

### BRIEF DESCRIPTION OF THE DRAWINGS

The drawings set forth herein are illustrative of exemplary embodiments provided herein and are not meant to limit the scope of the invention as encompassed by the claims.

The drawings are not done to scale, they are only for displaying some of the embodiments of the present invention.

FIG. 1 schematically illustrates an exemplary ultrasound endoscope (e.g., or colonoscope) with a one piece and/or very few pieces flexible ultrasound emitters diagonally wrapped around the endoscope's body.

FIG. 2 schematically illustrates an exemplary ultrasound endoscope with multiple flexible ultrasound emitters wrapped around the endoscope's body.

FIG. 3 schematically illustrates an exemplary ultrasound endoscope with ring transducers placed inside the endoscope's sheath at regular intervals.

FIG. 4 schematically illustrates an exemplary ultrasound endoscope with smaller sized ring transducers placed inside the endoscope's sheath with larger gaps between segments of ring transducers.

FIG. 5 schematically illustrates an exemplary wide beam ultrasound emitter array endoscope attachment in the closed position.

FIG. 6 schematically illustrates an exemplary wide beam ultrasound emitter array endoscope attachment in the open position.

FIG. 7 schematically illustrates an exemplary ultrasound endoscope with an irrigation and/or suction channel, a working channel, a camera and a plurality of disk-shaped ultrasound emitters placed inside the endoscope's sheath at regular intervals horizontally and longitudinally along the endoscope body up to nearly the endoscope's tip.

FIG. 8 schematically illustrates an overview of how an exemplary ultrasound endoscope operates when the ultrasound emitters are activated.

FIG. 9 schematically illustrates an exemplary ultrasound emitter array endoscope attachment, attached past the distal end of an endoscope

FIG. 10a schematically illustrates an exemplary ultrasonic waveguide (37) that is powered by an external ultrasound generator.

FIG. 10b a closeup of the distal part of an exemplary endoscope, showing where the ultrasonic waveguide situated inside a catheter, inserted into the endoscope' s working channel, is extended past the endoscope tip in order to vibrate and transfer its energy and acoustic waves to the surrounding liquid which transmits the acoustic waves to the tissue to be treated.

FIG. 11a schematically illustrates an exemplary ultrasonic waveguide and its tip, that is powered by an external ultrasound generator however the waveguide and ultrasound transducer are located inside the endoscope.

FIG. 11b schematically illustrates a close up of the distal part of an exemplary endoscope where the ultrasonic waveguide with a rounded tip connected via an attachment mechanism to the ultrasound transducer which is connected to the external ultrasound generator via a cable, extends past the endoscope tip in order to vibrate and transfer its energy and acoustic waves to the surrounding liquid which transmits the acoustic waves to the tissue to be treated.

FIG. 12a schematically illustrates an exemplary waveguide and its tip, that is powered by an external vibration control unit, however the waveguide and the vibrating motor are located inside the endoscope.

FIG. 12b a close up of the distal part of an exemplary endoscope (1) is shown where the waveguide with a rounded tip connected via an attachment mechanism to the vibrating motor which is connected to the external control unit via a cable, extends past the endoscope tip in order to vibrate in an oscillating motion and transfer its energy and acoustic waves to the surrounding liquid which transmits the acoustic waves to the tissue to be treated.

FIG. 13 schematically illustrates the distal part of a colonoscope with a confocal microscope array built into the colonoscope tip and an ultrasonic waveguide extended via the colonoscope's working channel to transmit the ultrasonic energy to the surrounding tissue. Spray holes and larger aspiration openings are situated proximally to the colonoscope tip.

FIG. 14 schematically illustrates the distal part of an exemplary colonoscope with a confocal microscope probe extended via the colonoscope's working channel to inspect for the presence or removal of biofilm from the surrounding tissue. Spray holes and larger aspiration openings are situated proximally to the colonoscope tip.

FIG. 15a schematically illustrates the lateral view of the distal part of an exemplary colonoscope with an overtube inserted over the colonoscope which encompasses spray and aspiration openings.

FIG. 15b schematically illustrates the frontal view of the distal part of an exemplary colonoscope with an overtube inserted over the colonoscope with one or more working channels built into the distal end of the overtube.

FIG. 16a schematically illustrates the lateral view of the distal part of an exemplary colonoscope with surface channels running along its length and a catheter inserted in each channel with the catheter having a plurality of spray and aspit=ration openings.

FIG. 16b schematically illustrates a three dimensional lateral view of the distal part of an exemplary colonoscope with surface channels running along its length to the colonoscope's tip.

FIG. 17 schematically illustrates the lateral view of an exemplary colonoscope with an oversized ultrasound emitter placed distally to the colonoscope's tip and a light source covering the distal part of the colonoscope. Also a surface channel with an embedded catheter having both spray and aspiration openings running the length of the colonoscope.

Like reference symbols in the various drawings indicate like elements.

### DETAILED DESCRIPTION

In alternative embodiments, provided are product of manufacture, uses and methods for the removal and/or destruction of gastro-luminal (e.g., colonic) biofilm, and including for the treatment or amelioration of biofilm-associated diseases, conditions and luminal infections, and devices and apparatus for the removal and/or destruction of gastro-luminal biofilm and for practicing methods as provided herein. In alternative embodiments, provided are devices and apparatus, and methods, for enhancing biofilm dissolving or disrupting agents, where in alternative embodiments the biofilm comprises a gastro-luminal ‘unstirred layer’, adherent layer or gastro-luminal mucus layer; or the biofilm comprises a matrix or a DNA-containing layer, or alternatively the biofilm comprises a polysaccharide gastro-luminal peripheral layer.

In alternative embodiments, provided are product of manufacture, uses and methods for the removal or treatment of infected biofilms, including infected biofilms in the gastrointestinal tract (GI), including the colon. In alternative embodiments, various devices and forms of equipment are attached to or can pass through (e.g., through the interior of) a product of manufacture, e.g., an apparatus, as provided herein. In alternative embodiments, provided are products of manufacture, e.g., an apparatus, comprising, and methods comprising use of, an ultrasonic device, which alternatively is designed to operate in a radial, longitudinal and/or omnidirectional mode, optionally using a vibrating waveguide.

In alternative embodiments, provided are products of manufacture, e.g., an apparatus or device, comprising, and methods comprising use of, lights of various wavelength, including comprising use of devices capable of producing, projecting and/or focusing lights of various wavelengths. In alternative embodiments, blue light, e.g., light in the range of about 470 nm, is projected by the product of manufacture, e.g., an apparatus or device, or an ancillary device used with the product of manufacture, e.g., project this light onto a biofilm to be disrupted or removed, or to be neutralized, e.g., sterilized.

In alternative embodiments, provided are products of manufacture, e.g., an apparatus or device, comprising, and methods comprising use of micro and/or nano-bubble-producing equipment.

In alternative embodiments, provided are products of manufacture, e.g., an apparatus or device, comprising, and methods comprising use of ozone-producing equipment, including use of ozonated liquids such as water, oils, and the like, and/or ozone gases which can contribute to destroy or disrupt, and subsequently remove, adherent biofilm.

In alternative embodiments, provided are products of manufacture, e.g., an apparatus or device, comprising, and methods comprising use of, mechanical equipment for the production of pulsating waves of liquids delivered to the GI tract, e.g., the colon, where in alternative embodiments the liquid being pulsated is a liquid biofilm-removing or biofilm disrupting formulation.

In alternative embodiments, products of manufacture, e.g., an apparatus or device, as provided herein are an improvement on an apparatus or machine which merely generates a liquid stream to be delivered via a speculum, a tube or the like inserted into the rectum (e.g., as a ‘colonic’ machine) to produce an enema or fecal washing or removal effect. These apparatus or machines are only designed to remove fecal matter and are not designed to and do not remove any biofilm or unstirred mucus layer.

In alternative embodiments, products of manufacture, e.g., an apparatus or device, as provided herein comprise all or components of any endoscope, gastroscope, nasal endoscope, bronchoscope, enteroscope, laparoscope, colonoscope, or any overtube which can be fitted to any of these devices, including devices used in the colon, stomach, small bowel or in any liquid-filled space or body cavity; and further comprise components that can deliver a ultrasonic power or pulse, and/or components that can deliver or effect non-inertial cavitation, inertial cavitation and/or microstreaming in situ, e.g., in a colonic lumen, and/or also delivering jets of water or other liquids, including delivery of the liquid with sufficient power or force to disrupt and/or remove an adherent biofilm.

In alternative embodiments, products of manufacture, e.g., an apparatus or device, as provided herein are positioned at a close distance to the surrounding tissue (e.g., a colonic luminal mucosa) to target or to deliver ultrasonic power, and thus effect an non-inertial cavitation, inertial cavitation and/or microstreaming.

In alternative embodiments, products of manufacture (e.g., devices or apparatus) as provided herein comprises or comprise use of an ultrasound generator, which is operatively connected to an ultrasonic probe or a waveguide; and when the ultrasound generator is turned on it drives an ultrasound emitter or waveguide to produce radial and/or longitudinal ultrasound waves perpendicular and/or omnidirectionally to the length of the devices or apparatus. In alternative embodiments, the ultrasound waves cause stable/non-inertial cavitation, inertial cavitation and/or microstreaming in the liquid (e.g., in the colonic lumen) surrounding the ultrasound device to remove and/or degrade materials attached or adherent to the tissue or target, such as a mucous-adherent biofilm, inspissated mucus, stool components, calcified material, or hardened canker-like materials not making up part of the living organism. The liquid surrounding the ultrasound device subjected to the stable/non-inertial cavitation, inertial cavitation and/or microstreaming may be blood, peritoneal fluid, water with or without added solutes, e.g., soap, urine, sinus fluid, bronchial fluid, lung fluid, cerebrospinal fluid, or other fluids capable of transmitting ultrasound waves. The liquid subject to the stable/non-inertial cavitation, inertial cavitation and/or microstreaming may comprise various substances, including for example, a biocide, or a biocide and ozone to increase the biocidal effect, e.g., against an infected biofilm, e.g., biofilm with bacteria within. In alternative embodiments, products of manufacture (e.g., devices or apparatus) as provided herein have components which can produce can produce micro and/or nano bubbles, and the externally added micro and/or nano bubbles can be added to the liquid mixture to enhance the stable/non-inertial cavitation, inertial cavitation and microstreaming effect.

In alternative embodiments, prior or during and/or after to the application of ultrasound in situ, e.g., to degrade, disperse and/or remove a biofilm, a pre-treatment, treatment or post-treatment protocol that comprises use of, or supplementation with, agents having an anti-biofilm (e.g., biofilm disrupting or dissolving) effect, and/or antibiotics (e.g., to combat infective organisms in the biofilm) are used. This supplemental reducing of biofilm and/or infective organism(s) (used with products of manufacture as provided herein, e.g., used with the ultrasound devices or waveguides as provided herein) can achieve better and longer lasting results of a desired therapy, for example, it can result in quicker, better and/or improved implantation of infused or ingested fecal microbiota transplantation (FMT) material, or improved treatment efficacies.

In alternative embodiments, provided are endoluminal devices comprising components or equipment adapted or designed to deliver to the bowels (e.g., intra-colonic luminal delivery) biofilm-destroying agents and/or biocides for enhancing the breaking up or dissolving of luminal biofilm, including infected, adherent biofilm.

In alternative embodiments, products of manufacture (e.g., devices or apparatus) as provided herein may also be fitted for (may also comprise components or attachments for) removing faecal matter or other debris from the gastrointestinal tract, e.g., the colon.

In alternative embodiments, products of manufacture (e.g., devices or apparatus such as endoscopic devices) and methods provided herein are (comprise) a biofilm treatment system that is delivered within the gastrointestinal tract, wherein alternatively the biofilm treatment can be delivered in situ or by a device, attachment or component attached to an endoscopic device, for example, are delivered by or via an overtube.

In alternative embodiments, products of manufacture (e.g., devices or apparatus such as endoscopic devices) and methods provided herein comprise use of components or attachments capable of generating sonic or ultrasound energy and/or vibrations or pulsations which, when transmitted in situ to the lumen of the GI tract, e.g., transmitted in situ to the lumen of a colon, can via a liquid interface generate sonic or ultrasound energy and/or vibrations or pulsations to disrupt or break up an adherent biofilm, or can enhance activity of a biocide, antibiotic or surfactant. In alternative embodiments, these effects are caused by or enhanced by the additional generation of nano/micro bubbles.

In alternative embodiments, products of manufacture (e.g., devices or apparatus such as endoscopic devices, or disposable sleeve(s) over the endoscope) comprise a plurality of spray holes, e.g., located towards the distal (interior) end of the endoscopic device, where alternatively the plurality of spray holes are located along an outer sleeve or equivalent that also can comprise tubes or sleeves or equivalents for transporting liquids or water under pressure. In alternative embodiments, the unstirred layer, or biofilm, removal or disruption is enhanced by mixing biofilm disrupting agents or compositions via the plurality of spray holes located along the length of the device or apparatus.

In alternative embodiments, after removal of the unstirred layer, or biofilm, in the GI tract, e.g., from the colon, a microbiome replacement is performed to implant a healthy microbiome, and thus a microbiome-derived biofilm, to protect the individual from a re-infection and to instigate a long-term remediation of symptoms.

In alternative embodiments, products of manufacture (e.g., devices or apparatus) comprise an ultrasound-producing module or attachment; a vibration- producing module or attachment; and/or, a waveguide-emitting module or attachment, where optionally the devices or apparatus are endoscopes, colonoscopes or probes operating in a radial, longitudinal and/or omnidirectional mode. In alternative embodiments, the products of manufacture can transmit ultrasound perpendicular to the device's or apparatus's (e.g., endoscope's or colonoscope's) body or distal tip. In alternative embodiments, in practicing methods as provided herein or when using products of manufacture as provided herein, the devices' or apparatus's (e.g., the endoscopes') body or distal tip is placed in a liquid-filled body cavity (e.g., a within a colon) in close proximity to the mucosal tissue, but in alternative embodiments is not apposing the mucosal tissue or is not touching the mucosal tissue.

In alternative embodiments, in practicing methods as provided herein or when using products of manufacture as provided herein, an ultrasound generator is turned on to drive an ultrasound emitter(s) to produce a radial, longitudinal and/or omnidirectional ultrasound waves along the length of the product of manufacture, for example, along the length of a flexible ultrasound endoscope or its distal tip. An ultrasound generator can be connected to the ultrasound emitter(s) via electrodes, cables or other means known in the art.

In alternative embodiments, an ultrasound emitter(s) is located on an outside layer of the product of manufacture, e.g., endoscope such as a colonoscope, and may or may not be covered by an ultrasound wave transparent sheath.

In alternative embodiments, products of manufacture (e.g., devices or apparatus) can be washed in the usual manner, for example, they can be washed, cleaned and/or sterilized as diagnostic ultrasound endoscopes are washed, cleaned and/or sterilized today.

In alternative embodiments, the ultrasonically active area, attachment or component of a product of manufacture (e.g., devices or apparatus, such as an endoscope or colonoscope) as provided herein can be a specific part length of the product of manufacture or is can be can extend the entire insertable (into the GI tract, e.g., colon) area and/or length of the product of manufacture (e.g., endoscope).

In alternative embodiments, the ultrasound wave or waves cause stable and/or non-inertial cavitation, inertial cavitation and/or microstreaming in a liquid surrounding the product of manufacture (e.g., endoscope or colonoscope), thereby enhancing removal and/or degradation of biofilm (e.g., adherent biofilm) from the tissue, e.g., mucosal tissue.

In alternative embodiments, in practicing methods as provided herein or when using products of manufacture as provided herein, a liquid surrounding the ultrasound product of manufacture (e.g., endoscope or colonoscope) comprises water and/or a saline, and optionally also comprises solutes or additives such as a soap, an oil or a biocide such as ozone. In alternative embodiments, water (such as e.g., distilled water, ozonized water (including super-ozonized water), hydrogen water, activated water, and/or electrolyzed water), saline, oils and/or other fluids are pumped thorough the product of manufacture into the surrounding tissue, e.g., into the surrounding tissue lumen. The biocide (e.g., ozone) can be used to increase a biocidal effect against a biofilm and any bacteria within it. Alternatively, the liquid can comprise an oil (e.g., mineral oil) of any kind, particularly one that readily transmits ultrasound waves.

In alternative embodiments, the ozone concentration in the liquid can range from between about 0.1 parts per million to about 10 parts per million; or the ozone concentration in the liquid can range from between about 0.1 part per million to 7 parts per million.

In alternative embodiments, the fluid or liquid surrounding the acoustic and/or ultrasound product of manufacture (e.g., endoscope or colonoscope), or the fluid or liquid pumped through the product of manufacture and into the surrounding tissues or lumen space, comprises: surfactants, soaps, detergents, wetting agents, emulsifiers (e.g., carboxymethylcellulose or polysorbate or ceteareth (e.g., polyoxyethylene ethers of a mixture of high molecular mass saturated fatty alcohols, such as cetyl alcohol and stearyl alcohol), foaming agents, lecithins, esters of monoglycerides of fatty acids, or mono- and/or diglycerides of fatty acids, and/or dispersants, which can be used to reduce the surface tension of the liquid and/or to reduce its cavitation threshold.

In alternative embodiments, the fluid or liquid surrounding the acoustic and/or ultrasound product of manufacture (e.g., endoscope or colonoscope), or the fluid or liquid pumped through the product of manufacture and into the surrounding tissues or lumen space, which will be the liquid or fluid surrounding the acoustic/ultrasound component or attachment (e.g., of the endoscope), can be saturated or supersaturated with oxygen or carbon dioxide gas, e.g., for the purpose of lowering the surface tension and cavitating threshold of the liquid or fluid medium; this results in requiring less energy to induce stable/inertial cavitation and more pronounced acoustic streaming (see e.g., Yamashita, T., & Ando, K. Low-intensity ultrasound induced cavitation and streaming in oxygen-supersaturated water: Role of cavitation bubbles as physical cleaning agents. Ultrasonics Sonochemistry; Hauptmann, Marc & Frederickx, F & Struyf, Herbert & Mertens, Paul & Heyns, Marc & De Gendt, Stefan & Glorieux, Christ & Brems, Steven. (2012). Enhancement of cavitation activity and particle removal with pulsed high frequency ultrasound and supersaturation. Ultrasonics sonochemistry).

In alternative embodiments, products of manufacture (e.g., devices or apparatus) are fabricated as an endoscope or endoscope-like device, e.g., an colonoscope, or an overtube. In alternative embodiments, endoscope or endoscope-like device further comprises an overtube. In alternative embodiments, the overtube is designed or fabricated as an elongated flexible tube that has a proximal and distal end, and which can be of various lengths and diameters, and has a channel within to pass a liquid or a fluid (optionally under pressure) along the length of the elongated flexible tube, and the liquid or fluid can eventually pass into a body cavity, e.g., a lumen of a colon, optionally the liquid or fluid passes from the channel in the overtube or a product of manufacture as provided herein through a plurality of opening along the overtube or product of manufacture, wherein optionally the plurality of opening along the overtube or product of manufacture are positioned towards the distal end of the overtube or product of manufacture. In alternative embodiments, products of manufacture with overtubes, or the overtubes as provided herein, also comprise an additional channel to suction out the liquid from the body cavity. The channel passing the liquid or fluid to the body cavity and the channel carrying or suctioning the liquid or fluid out of the body cavity can be the same or can be two different channels. In alternative embodiments, the length of the insertable (e.g., into the colon) part of the product of manufacture (e.g., endoscope) ranges from between about 3 centimeters to about 3500 centimeters (3.5 m). In alternative embodiments, the diameter of the product of manufacture (e.g., endoscope) can range from between about 1 millimeter to about 5 centimeters.

In alternative embodiments, products of manufacture (e.g., devices or apparatus) comprise one or a plurality of external or internal layers, where the external or internal layers have lumens or passages through which one or more components or tubings can be passed, for example, these lumens can carry (or allow passage through) one or more ultrasound emitting elements (e.g., a plurality of ultrasound emitter arrays), where the ultrasound emitting elements can emit ultrasound energy in the longitudinal and/or the radial mode.

In alternative embodiments, products of manufacture (e.g., devices or apparatus) can be covered with an appropriate sheath or equivalent to protect the components or tubings, e.g., to protect the emitting elements (e.g., a plurality of ultrasound emitter arrays), and to permit washing of the instrument.

Alternatively, the one or more components or tubings, e.g., ultrasound emitting elements (e.g., a plurality of ultrasound emitter arrays), are each housed in a separate disposable or reusable sheath such as the kind slipped onto a standard instrument such as a bronchoscope, gastroscope or colonoscope. In another embodiment, the ultrasound emitters are exposed to the liquid medium and washed afterwards as per the current methods for ultrasound endoscopes.

In alternative embodiments, the emitted ultrasound energy travels to the tissue being treated via a liquid interface, and thus creates stable cavitation, inertial cavitation and microstreaming along the tissue wall (e.g., colonic mucosal wall); thus, an adherent biofilm on the tissue can be removed and/or degraded.

In alternative embodiments, externally added stabilized nano/micro bubbles are added to the liquid being administered (e.g., passed through a product of manufacture (e.g., device or apparatus) to enhance the “cleaning” action of the product of manufacture.

In alternative embodiments, a camera is built into the product of manufacture (e.g., endoscope or colonoscope) to allow the operator to guide the product of manufacture to the area to be treated. The product of manufacture (e.g., endoscope) can be manually maneuvered around the body cavity being treated (e.g., colon) in order to move it closer to the tissue if needed, for example, to ensure that the administered ultrasound energy is effectively reaching an area of interest, and thus causing stable/non-inertial cavitation, inertial cavitation and/or microstreaming, thus dissolving and/or degrading a biofilm, e.g., an adherent biofilm.

In alternative embodiments, an ultrasound or other ‘stirring’ device array is fabricated as part of the distal part (or end) of the product of manufacture (e.g., endoscope). The ultrasound array can be part of the product of manufacture (e.g., ultrasound endoscope) or it can be an attachable (and thus removable) part to the product of manufacture.

In alternative embodiments, the ultrasound array is a wide beam array, or it can transmit ultrasound in a focussed or non-focussed fashion.

The ultrasound array can be a one or several use attachments; and can use replaceable component(s) or reusable part(s) that can be re-used after appropriate reprocessing and/or cleaning (including sterilization) has taken place, as known to those in the art.

In alternative embodiments, a plurality of ultrasound emitter arrays, e.g., two, three or four or more, are (e.g., optionally removably) attached around the product of manufacture (e.g., endoscope).

In alternative embodiments, the ultrasound arrays have a curvilinear shape and smooth edges all around, e.g., to allow easy insertion and extraction from the body and to prevent causing any injuries once inside the bodily cavity; or, to facilitate movement thorough a sleeve or overtube attachment to a product of manufacture as provided herein; or, to facilitate movement thorough a separate disposable or reusable sheath; or, to facilitate movement thorough external or internal layers of products of manufacture as provided herein, wherein the external or internal layers have lumens or passages through which the ultrasound arrays can pass.

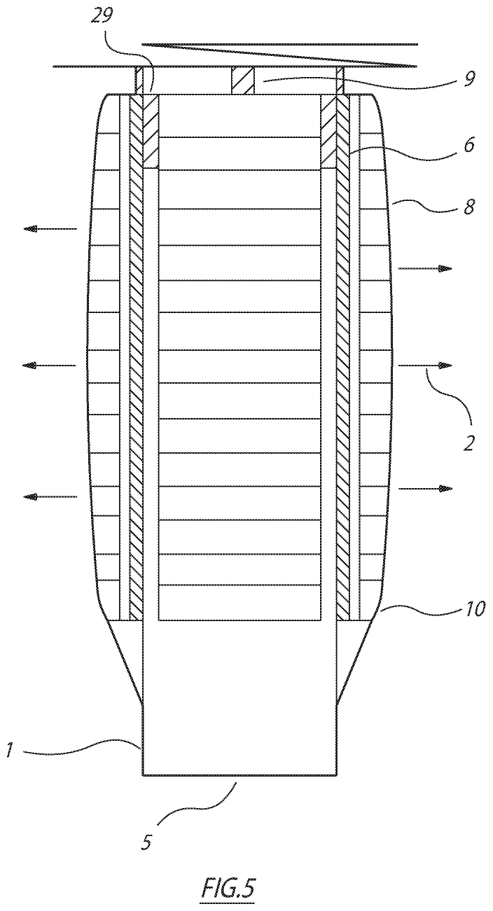

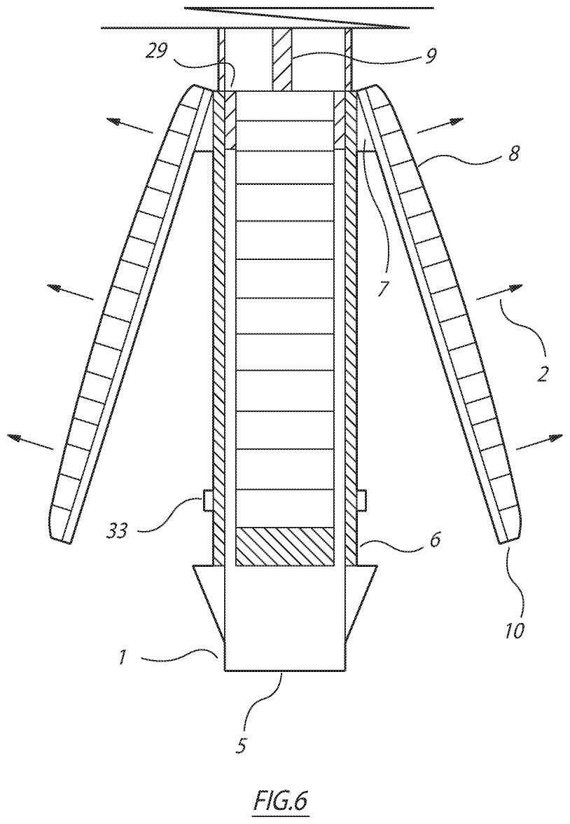

In alternative embodiments, upon insertion as an attachment on the distal end of the colonoscope and when the product of manufacture (e.g., endoscope or colonoscope) has reached an area to be treated, the ultrasound array or arrays are released (e.g., manually, e.g., by a triggering mechanism) by the treating physician so that each ultrasound array is released from the product of manufacture (e.g., endoscope) and sits up; e.g., an array can sits on top of the endoscope as an attachment and it can either stay flat against the endoscope or can be released and each section lifts and rises from surface as illustrated in FIGS. 5 and 6. In alternative embodiments, the triggering mechanism is part of a handling mechanism of the product of manufacture (e.g., endoscope) or it can be a separate apparatus. The triggering mechanism can be connected to the ultrasound array by a cable or cables, or it can initiate a releasing action remotely by transmitting a signal to the ultrasound array inside the body cavity.

In alternative embodiments, each ultrasound array is released from its distal part so that each array's active ultrasound emitters rise from their distal end being raised by a spring mechanism at the array's proximal end and face back (e.g., the release of the distal part of the array, e.g., a spring mechanism at the proximal end lifting it up, as illustrated in FIGS. 5 and 6) towards the proximal part of the endoscope.

In alternative embodiments, ultrasound array emitters are situated, positioned, or optionally removably attached, at any point circumferentially to the length of the endoscope.

In alternative embodiments, the arrays' angle to the length of the product of manufacture (e.g., endoscope) (e.g., the angle between the flat surface of the colonoscope and the array being released and rising from its distal part) can range from 0 degrees to 90 degrees. The ultrasound arrays can transmit ultrasound at between 0 degrees and 180 degrees.

In alternative embodiments, a mechanism or attachment holding an ultrasound transducer raised up is pliant or flexible in order to allow extraction from the body once it reaches a body opening, e.g., a rectum. When the body opening is reached the tissue pressing against the raised wide beam transducers will push them down against the endoscope. The mechanism can be spring loaded, or hydraulically or pneumatically operated, or can use any known mechanism. For example, when it reaches the rectum, the array can go back to its closed position from the pressure exerted on it (push it closed) to allow the colonoscope and attached array to be removed from the colon; this is why only the distal part can be raised to allow the raised arrays to be pushed closed when the device comes across a tight area.

In alternative embodiments, the ultrasound array, probe or attachment can transmit ultrasound from between about 10 KHz to about 30 MHZ, or from between about 5 KHz to 60 MHZ.

In alternative embodiments, the transmitted ultrasound frequency can be one specific frequency or it can modulate between a range in of frequencies, e.g., can modulate between a range from about 10 KHz to about 30 MHZ. The modulated frequencies can be: emitted in a CHIRP (Compressed High-Intensity Radiated Pulse); emitted in a Periodic Selection of Random Frequency (PSRF) fashion; be a sweep up, sweep down, sweep up/down or sweep down/up emission; or, any combination of these modulation approaches. In alternative embodiments, the transmitted ultrasound can be in a continuous or pulsed mode and of fluctuating amplitudes.

In alternative embodiments, the power output of the ultrasound emitters ranges from 0.001 Watt per square centimetre to 400 Watts per square centimetre. In alternative embodiments, the power output of the ultrasound emitters ranges from 0.01 Watt per square centimetre to 200 Watts per square centimetre. These intensities can be: Spatial Peak-Temporal Peak (SPTP); Spatial Average-Temporal Peak (SATP); Spatial Average-Temporal Average (SATA); Spatial Peak-Pulse Average (SPPA); or, Spatial Average-Pulse Average (SAPA) intensity types.

In alternative embodiments, an ultrasound emitter is part of the distal tip of a product of manufacture (e.g., endoscope or colonoscope) as provided herein, or the ultrasound emitter can be (e.g., optionally removably) attached to product of manufacture's (e.g., an endoscope's) distal tip.

In alternative embodiments, the ultrasound emitter is arranged in an array, e.g., as a linear, convex, phased, omnidirectional or radial array. The array can emit ultrasound in a field between 0 to about 360 degrees. In alternative embodiments, an emitter in this configuration may also emit ultrasound in a forward-facing manner.

In alternative embodiments, in practicing methods or products of manufacture as provided herein, pre-treatments can be given to a patient, for example, an oral pre-treatment comprising administration of an antibiotic and/or an anti-biofilm (e.g., disrupting or dissolving) supplement. In alternative embodiments, anti-biofilm supplement pre-treatments can be one or combinations of any of the following: N-Acetyl Cysteine, bismuth and bismuth analogues, alginate and alginate analogues, soap and water, catechins and epicatechins like Epigallocatechin gallate, Ethylenediaminetetraacetic acid (EDTA), alpha lipoic acid, mesalazine, sulfasalazine and their analogues and other agents known in the art to have an anti-biofilm effect. In alternative embodiments, antibiotics can comprise, or can be selected from, any of the following: tetracycline, doxycycline, tobramycin, minocycline, penicillin and its derivatives for example amoxicillin, metronidazole, carbapenem and its derivatives, gentamicin, secnidazole, furazolidone, nitazoxanide, paromomycin, iodoquinol and other antibiotics and combinations thereof.

In alternative embodiments, the pre-treatment period is between about one (1) to two (2) weeks, or the pre-treatment period can last as long as several weeks (e.g., 2 to 10 weeks) or months (e.g., 2 to 12 months). In alternative embodiments, anti-biofilm supplements are given to a patient in an enterically coated capsule so the active ingredients are released in the small intestine and/or large intestine. Soap-containing capsules releasing in the distal small bowel also are used to pre-damage a colonic biofilm.

In alternative embodiments, a sonic, acoustic wave or an ultrasonic wave energy can reach the biofilm location in the GI tract, e.g., the colon, via the use of an acoustic waveguide. In one embodiment, the waveguide is of a uniform thickness and cross-sectional area from its proximal end attached to the transducer, or to its distal tip. In another embodiment, the waveguide is of a variable thickness and cross-sectional area starting with a larger cross-sectional area at its proximal end attached to the transducer and progressively getting thinner towards its distal end. By tapering the cross-sectional area, the vibration amplitude of the acoustic wave transmitted via the waveguide is amplified and results in a larger amplitude displacement at the tip (or distal end), resulting in a more powerful emitted acoustic field. In alternative embodiments, an acoustic waveguide can have two, three or more tapered sections to enable amplification of the vibratory displacement of the tip.

In alternative embodiments, the tip (or distal end) at the end of the acoustic waveguide can have a diameter of between about 0.5 mm and 30 mm. In alternative embodiments, the shape of the tip can have various different geometries, for example, it can be shaped like a semi spherical tip, spherical tip, a cylinder tip, a square tip, a pear-shaped tip or a flat tip. In alternative embodiments, the length of the waveguide can be between 1 cm to 3500 cm. The amplitude of the tip depends on the power that is transmitted through it; so, in alternative embodiments, the displacement amplitude of the tip is between about 1 μm to 1000 μm peak-to-peak.

In alternative embodiments, the acoustic waveguide is manufactured using a metal that can transmit sonic and ultrasonic waves; for example, the waveguide can be fabricated using aluminium and its alloys, titanium and its alloys, nickel titanium (nitinol) and stainless steel and its alloys, or any other metals.

In alternative embodiments, the acoustic waveguide is placed into the product of manufacture (e.g., an endoscope or colonoscope) working channel via a catheter, tube or sleeve to prevent damage to the product of manufacture. The catheter or sleeve can be made of plastic, nylon, a polymer, flexible metal or any other flexible material.

In another embodiment, the location of the transducer driving the acoustic waveguide is placed near the tip (or distal end) of the product of manufacture (e.g., endoscope or colonoscope) to minimize the acoustic wave's loss due to the distance travelled. In alternative embodiments, a short length acoustic waveguide is (e.g., optionally removably) attached to the transducer, and the product of manufacture is inserted into the body cavity to be treated (e.g., colon). In this embodiment, the acoustic waveguide can have a length of between about 0.1 cm and 10 cm; or, the waveguide can have a length of between about 1.0 cm and 8 cm.

In another embodiment, the tip (or distal end) of the product of manufacture (e.g., endoscope or colonoscope) houses or has (e.g., optionally removably) attached thereto a motor can vibrate, e.g., can vibrate at between about 50 Hz to 10000 Hz. In alternative embodiments, an acoustic waveguide is (e.g., optionally removably) attached to the vibrating motor and the product of manufacture is inserted in the body cavity to be treated. In this embodiment, the waveguide does not have to vibrate ultrasonically; however, a rapid oscillating movement of the tip (or distal end) is sufficient to cause biofilm degradation and/or dispersal, or cause disruption of a mass if that is the objective. In this embodiment, the acoustic waveguide can have a length of between about 0.1 cm and 10 cm, or between about 1.0 cm and 8 cm.

In alternative embodiments, the acoustic waveguide material comprises or is manufactured using a metal, a plastic, a polyamide, an elastomer, a polymer or any combination of materials. For example, the main body of the wave guide can be made out of nitinol and the tip (or distal end) made of polyamide. The end of the waveguide can have a diameter of between about 0.5 mm and 30 mm, or 5 mm and 25 mm.

In alternative embodiments, the shape of the acoustic waveguide tip (or distal end) can have various different geometries like a semi spherical tip, spherical tip, a cylinder tip, a square tip, a pear-shaped tip or a flat tip. In alternative embodiments, the waveguide tip is covered in soft bristles to enhance the hydrodynamic action of the tip and, if present, the mixing of the nano/micro bubbles, in the vicinity of the gastrointestinal mucosa (e.g., colonic mucosa) to enhance the biofilm removal or disruption action.

In alternative embodiments, the acoustic waveguide is positioned in the location to be treated via the product of manufacture's (e.g., the endoscope's) working channel, e.g., an internal or external lumen or sleeve. However, because some endoscopes do not have a large enough working channel to accommodate the diameter of an acoustic waveguide, in some embodiments, the product of manufacture comprises or comprises use of an overtube or a sleeve or equivalent with a channel or lumen built into it that is large enough to accommodate the acoustic waveguide, thus allowing this therapy to be used with most types of endoscopes.

In alternative embodiments, a product of manufacture (e.g., endoscope) as provided herein comprises or comprise use of an overtube or sheath placed over the product of manufacture, and the overtube or sheath has a plurality of water jets and/or spray holes placed around its circumference projecting a liquid stream out or away from the product of manufacture in a straight or angled direction. In alternative embodiments, the plurality of water jets and/or spray holes begin at the product of manufacture's (e.g., endoscope's) tip (or distal end) and extend backwards or more proximally, and optionally at the same or increasing distance between the liquid dispensing holes. In alternative embodiments, there are between about 1 to 50, or 1 to 100, spray holes; and these can be placed in line along the product of manufacture (e.g., endoscope) from between about 1 centimeter (cm) to about 100 cm of the product of manufacture's tip (or distal end). The spray holes can be arranged in one, two, three, four or more lines along the product of manufacture (e.g., endoscope), or they may be circularly positioned, e.g., in order to wash the instrument during the procedure and/or to lubricate the area by spraying the liquid on the surrounding mucosa so the product of manufacture (e.g., endoscope) can be more easily maneuvered. In alternative embodiments, the stream is not used for removal of feces, but rather for the removal of broken-up or dissolved biofilm. In alternative embodiments, the spray holes are in a circular pattern, a zig zag pattern or a random pattern around the product of manufacture.

In alternative embodiments, the product of manufacture (e.g., endoscope), or the overtube, sheath or sleeve or equivalent, also has a large suction channel to remove liquid or fluids at the same or close to the same rate as the liquid or fluids are irrigated into an area (e.g., a body cavity) to be treated (e.g., the colon) after the body cavity has been filled with the liquid or fluids. In alternative embodiments, the combined action of irrigation and suction of the liquid or fluids causes mixing of the liquid or fluids in the body cavity, thus enhancing the effect of the liquid or fluids breaking up or dissolving the biofilm present on (e.g., adherent to) a tissue (e.g., mucosal) surface.

In alternative embodiments, at the tip (or distal end) of the product of manufacture (e.g., endoscope), there is a line of (e.g., a plurality of) aspirating openings situated next to the spray holes that remove any dissolved material (such as biofilm debris), particularly from the cleaning surfaces of the product of manufacture. These aspiration openings can be larger than the spraying holes as to allow effective and rapid removal of any debris. The spray holes and/or the aspirating openings can have a diameter of between about 0.2 millimeters (mm) to 10 mm, or between about 2 mm to 7 mm, or between about 1 mm and 5 mm.

In alternative embodiments, a liquid or fluid is sprayed and/or aspirated by using an overtube or sleeve or equivalent attached around the product of manufacture (e.g., endoscope or colonoscope); and the overtube or sleeve or equivalent can be used in a one use or repeated use fashion after appropriate reprocessing (e.g., cleansing, sterilization) has taken place. In this embodiment the overtube or sleeve or equivalent can have a multiple purposes, for example: causing mixing of the liquid or fluid present in the body cavity (e.g., colon), for example, mixing the liquid or fluid sprayed into the body cavity by the product of manufacture); keeping the product of manufacture clean during the procedure; enhancing the product of manufacture's ease of insertion into the body cavity (e.g., the colon); and, enhancing the product of manufacture's manoeuvrability by lubricating the surrounding mucosa and device itself

In alternative embodiments, the irrigation and suction channel(s) are connected to a pumping and aspirating unit; this unit can be situated proximally to the product of manufacture (e.g., endoscope), and the unit also can measure and/or regulate irrigation flow, pressure, temperature, irrigation/suction channel selection, section or sections of activated spraying, aspirating openings or any combination thereof and, all these parameters can be adjusted using the controls on the unit, and all these parameters can be displayed on monitors or display modules on the unit.

In alternative embodiments, the product of manufacture (e.g., endoscope) has a confocal microscope, a microscope probe, an optic microscope or equivalent (any probe-based microscope or visualizing apparatus) (optionally removably) attached at its distal end, and the image of tissues created by the confocal microscope or equivalent can be used to scan for biofilm-covered areas on a tissue being treated, e.g., on a colonic mucosa. The confocal microscope or equivalent can be controlled remotely by an operator, and the confocal microscope or equivalent can be set to scan continuously, intermittently or only when the operator believes an area of biofilm covered tissue has been encountered; and biofilm covered areas can be identified by either increased inflammation or by a biofilm-revealing stain that has been injected or irrigated into the area previously to stain and reveal any biofilm-covered areas in the body cavity (e.g., adherent on the mucosa) being treated, which can use fluorescence and/or UV light. The confocal microscope or equivalent can be used before, during or after the biofilm dissolution or biofilm reducing procedure to check for any biofilm or biofilm remnants and to decide whether additional treatment is required.

In alternative embodiments, the confocal microscope probe, optic microscope or equivalent is inserted into a product of manufacture as provided herein via a wide working channel to carry out in situ visualization of tissues.

In alternative embodiments, the confocal microscope probe, optic microscope or equivalent has zooming capabilities that allow visualization of tissues of interest, e.g., for closeup visualization of a stained biofilm in vivo during the procedure. In alternative embodiments, the confocal microscope probe, optic microscope or equivalent has resolution and z-depth in the tens to hundreds of nanometres; and/or has a zoom capacity of 10 to 500 times magnification. The optical zoom can be between about 50 times magnification to about 100 times magnification, or greater.

In alternative embodiments, the confocal microscope probe, optic microscope or equivalent is proximally connected to a unit which controls its functions and a screen which displays a real time image of the tissue being analysed by the confocal microscope probe, optic microscope or equivalent to assist the operator in successfully carrying out a procedure, e.g., a biofilm removal or disruption. The unit can have an artificial intelligence-assisted program to assist the operator in identifying biofilm and tissue affected by biofilm in order to reduce the time of the procedure and to enhance its effectiveness.

In alternative embodiments, products of manufacture (e.g., endoscopes, gastroscopes, enteroscopes, colonoscopes, sigmoidoscopes) as provided herein can have a large diameter working channel to allow for the insertion of: an ultrasonic probe, e.g., to degrade the tissue-attached (e.g., adherent) biofilm; a confocal microscope probe, optic microscope or equivalent to, e.g., check for the presence or removal of the biofilm, and/or for the removal of large polyps to permit easier passage of large polyps down the working channel; an irrigation device; an liquid or fluid evacuating device; an illuminating device; and/or, any other device or apparatus. In alternative embodiments, the working channel is between about 1 to 20, 2 to 10, or 3 to 5 millimetres (mm) in diameter.

In alternative embodiments, products of manufacture (e.g., endoscopes, gastroscopes, enteroscopes, colonoscopes, sigmoidoscopes) as provided herein address limitations of known ultrasound cleaning devices; for example, where known ultrasound cleaning devices rely mostly on longitudinal waves emanating from an ultrasound tip at the distal end of an apparatus to deliver the ultrasound to the target tissue under a flowing fluid stream, e.g., an acoustic coupler as in dentistry; or, because of the small size of probes in known ultrasound cleaning devices the area treated is usually small and the management of the flowing liquid coupler in an internal body cavity is challenging, e.g., as in a clinical setting of the gut lumen. These drawbacks of known ultrasound cleaning devices make them inefficient when trying to treat larger tissue surface areas, for example, when attempting to clean or debride colonic mucosa, or the large intestine wall.

Thus, in alternative embodiments, products of manufacture (e.g., endoscopes, gastroscopes, enteroscopes, colonoscopes, sigmoidoscopes) as provided herein addresses these problems by having embedded a single or a plurality of ultrasound emitters on their sheath, distal tip or attachment, such as an over-tube sleeve fitting. The plurality of ultrasound emitters can be spaced between about 0.5 cm to 10 cm apart. The single or plurality of ultrasound emitters can emit ultrasound: in a radial and/or longitudinal mode, perpendicular to the length of the endoscope or omnidirectionally.

In alternative embodiments, products of manufacture (e.g., endoscopes, gastroscopes, enteroscopes, colonoscopes, sigmoidoscopes) as provided herein, including over-tube sleeve fittings and equivalents, having large or jumbo channels, are designed to have inserted therein cleaning devices, e.g., stool-cleaning device such as a PURE-VU stool-cleaning device (Motus GI Holdings, Inc., Fort Lauderdale, Fla.). By incorporating a stool-cleaning device, products of manufacture as provided herein can efficiently clean a liquid-filled cavity such as the stomach, small bowel or colon; and relatively large surface areas can be simultaneously cleaned and treated, thus making any procedure much more time efficient and effective. For example, in alternative embodiments, a larger ultrasonic probe can be inserted via a jumbo biopsy channel in a product of manufacture (e.g., endoscope) as provided herein, and the ultrasonic probe can be moved within the (e.g., colonic) lumen in conjunction (in coordination) with the directed movement (optionally under direct visualization by an operator) to deliver a biofilm-dissolving or disrupting agent, liquid and/or fluid to large surfaces of the gut.

In alternative embodiments, methods as provided herein, and/or products of manufacture as provided herein, use or provide (e.g., deliver) a liquid or fluid medium to the body cavity (e.g., colon) in sufficient amounts that the ultrasound energy (e.g., provided by an ultrasound array of a product of manufacture as provided herein) can propagate via or through the liquid or fluid to create a stable and/or non-inertial cavitation, an inertial cavitation and/or microstreaming along the tissue (e.g., mucosal) wall in order to degrade and/or remove a biofilm target. The bowel can be filled with water or various other liquids, including those which are capable of dissolving or disrupting a biofilm (including biofilm matrix components). The liquid or fluid medium can comprise saline, activated water, ozonized water, electrolyzed water, distilled water, molecular hydrogen-rich water, saline or water with various additives, hyperoxidized water, distilled water, soap and water, molecular hydrogen-rich water, iodine or iodine-containing liquids, a super-oxidized solution (SOS) (also known as anolyte solution and oxidative potential water) such as MICROCYN™ or MICRODACYN™, PHMB (poly hexa methylane bioguanine or derivatives), ozone gas or ozonated water, and/or any combination thereof.

In alternative embodiments, a liquid or fluid stream is introduced into the tissue under pressure at the tip of the apparatus in order to transfer the ultrasound energy to the tissue, and to also induce cavitation in the liquid. In alternative embodiments, a liquid or fluid stream is introduced into the body cavity under low or no pressure, and can be slowly introduced to fill a body cavity (e.g., a colon) and surround the tip, some of the length of, or all the length of, a product of manufacture as provided herein, e.g., a flexible endoscope. In alternative embodiments, the liquid or fluid stream is introduced in a pulsating manner or mode to enhance cleaning.

In alternative embodiments, methods and products of manufacture (e.g., endoscopes, gastroscopes, enteroscopes, colonoscopes, sigmoidoscopes) as provided herein, deliver ultrasound via one or a plurality of ultrasound emitters, which can be embedded into the external wall or the distal tip of the products of manufacture.

In alternative embodiments, methods and products of manufacture as provided herein can be flexible devices, e.g. An endoscope, inserted into the body cavity, and the ultrasound can be delivered in a mode or direction perpendicular, radially and/or omnidirectionally to the length of the product of manufacture, or along the length of the product of manufacture, and/or omnidirectionally to the distal end or tip of the products of manufacture.

In alternative embodiments, methods and products of manufacture as provided herein provide liquids or fluids comprising bioactive agents, e.g., bio-film disrupting agents, biocides or antibiotics, or other compounds such as a biofilm or a tissue stain. In alternative embodiments, a body cavity is filled with a liquid or fluid (e.g., comprising a biocide or antibiotic), and alternatively the liquid or fluid is not delivered under pressure.

In alternative embodiments, methods and products of manufacture as provided herein provide sufficient liquids or fluids to a body cavity (e.g., to a colon) to transmit the ultrasound produced by the ultrasound array, thus dislodging the tissue-adherent biofilm. In alternative embodiments, the liquids or fluids are added or delivered prior to initiation of ultrasound therapy to the liquids or fluids can act as a medium to effectively transfer the ultrasound to the tissue and to induce stable/non-inertial cavitation, cavitation and/or microstreaming in the liquid to e.g., dislodge or disrupt a biofilm.

In alternative embodiments, methods and products of manufacture as provided herein provide add nano- and/or micro-bubbles to the liquid or fluid; this adds to the effectiveness and efficiency of the liquid or fluid to remove the biofilm, e.g., adds to the effectiveness and efficiency of the stable/non-inertial cavitation, cavitation and/or microstreaming effect to remove the biofilm.

In alternative embodiments, methods and products of manufacture as provided herein comprise use of or deliver in situ: biocides, e.g., biocides such as a water (e.g., ozonized water, distilled water, activated water, electrolyzed water or other waters, e.g., as described herein); antiseptics safe for in situ use; and/or antibiotics, or any combination thereof

In alternative embodiments, methods and products of manufacture as provided herein comprise use of or deliver in situ: liquids or fluids saturated or supersaturated with oxygen, carbon dioxide or another biocompatible and safe gas to reduce the surface tension and/or cavitation threshold of the liquid medium.

In alternative embodiments, methods and products of manufacture as provided herein deliver ultrasound via one or a plurality of ultrasound emitters embedded into the external wall of a product of manufacture as provided herein, e.g., a flexible endoscope or probe, which, when inserted into a bodily cavity (e.g., the colon), the ultrasound is delivered in a mode/direction perpendicular to the axis of the product of manufacture as provided herein.

In alternative embodiments, when using products of manufacture as provided herein, they are contacted with the tissue wall; in this embodiment the ultrasound is transferred to a tissue (e.g., a colon mucosa) via a liquid or fluid interface, which can be introduced in the bodily cavity to be treated, and sufficient amounts can be introduced to fully surround the ultrasonically active area of the products of manufacture, e.g., as flexible endoscopes and/or probes.

In alternative embodiments, methods and products of manufacture as provided herein comprise use of a three- or four-step approach to disrupting and/or removing biofilm (e.g., mucosa-adherent biofilm) in the body:

First: methods as provided herein begin with a systemic antibiotic and/or anti-biofilm oral pre-treatment, which may start, one or two to several weeks before mechanical treatment. This pre-treatment is designed to reduce the bacterial load before the mechanical treatment, and to target bacteria residing in the mucosal and intra-mucosal regions and other areas of the body like the appendix, these bacteria if not completely killed or removed can cause re-infection after the mechanical treatment is completed.

Second: during mechanical treatment ultrasound, pulsation or sonic waves are used to degrade, disrupt and/or remove a biofilm matrix, e.g., a mucosal-adherent biofilm.

Third: inactivating or killing biofilm-encased bacteria with the use of biocides and/or antibiotics mixed with the liquid or fluid medium transmitting the acoustic waves to the biofilm, where the liquid or fluid medium is delivered by a product of manufacture or an attachment or ancillary device to a product of manufacture as provided herein, as discussed above.

Fourth: and optionally, immediately or shortly after step 3 is carried out, introducing a new, healthy or therapeutic microbiota into the bowel for colonization to replace the infected biofilm, thus maximizing the chances of long-term improvement in the patient's health and effective treatment of a disease or condition caused or exacerbated by the infected biofilm. Simply removing or partially degrading the biofilm, and optionally killing all or most of the biofilm-released bacteria does not ensure that remaining or newly introduced pathogenic bacteria will not create a new pathogenic biofilm which will eventually bring back or cause recurrence of the same symptoms, disease or condition. With the introduction of the new or therapeutic microbiota immediately after removal of the biofilm, a new healthy biofilm can be rapidly recreated, and also can possibly eliminate any remaining pathogenic bacteria by releasing antimicrobial substances such as bacteriocins and via competitive exclusion. Introduction of a new or therapeutic microbiota, can be done either as a slurry delivered via an endoscope, or in capsules, which can be suppositories or can be consumed orally.

In alternative embodiments, methods and products of manufacture as provided herein comprise use of ultrasound endoscopes or components as described by e.g., U.S. Pat. No. 6,238,336; JP3709325B2; JP2005118133A; JP4526298B2; JP3722667B2; U.S. Pat. No. 9,398,843; EP2596753B1; U.S. Pat. No. 7,318,806, and US patent publications 2019/0111130 A1; 2008/0051655 A1; 2006/0009681 A1; and 2013/0253387 A1, some of which describe ultrasound endoscopes whose purpose is solely for visualisation and observation purposes, with an ultrasound transducer placed at the distal tip of the endoscope.

In alternative embodiments, methods and products of manufacture as provided herein transmit ultrasound waves perpendicular, radially and/or omnidirectionally to the length of the product of manufacture (e.g., endoscope and/or probe) in order to achieve a therapeutic effect, and merely for visualisation and/or diagnostic purposes. In alternative embodiments, methods and products of manufacture as provided herein transmit waves over a large area of the product of manufacture, for example, waves are transmitted throughout its length, over the distal half of its length, or over the distal quarter of its length, and not just at the distal end or tip, or over between about 20% to 100%, or 30% to 90%, of its length.

In alternative embodiments, methods and products of manufacture as provided herein transmit ultrasound waves in a frequency range of between about 10 KHz to 30 MHz, or 5 KHz to 40 MHz, or more (ultrasound endoscopes used for visualisation or diagnostic purposes operate in the 2 to 20 MHz range).

In alternative embodiments, methods and products of manufacture as provided herein are designed for the dissolution of a biofilm matrix, e.g., a mucosa-adherent biofilm, which encapsulates or surrounds and protects the bacteria residing inside it. In alternative embodiments, an important purpose of the antibacterial and/or biocide substance or substances included in the introduced liquid or fluid medium is to reduce the possibility of the bacteria landing at another site and setting up a new pathogenic biofilm colony. In alternative embodiments of methods as provided herein, luminal fluids or liquids are exchanged several times (e.g., 2, 3, 4, 5 or 6 or more times) to more completely remove a fractionated, disrupted and/or damaged biofilm; and after each removal cycle the body cavity (e.g., colonic lumen) is resupplied with fresh liquid or fluid for subsequent treatment or treatments. Use of the combination of the ultrasound and antibacterial treatments as provided herein creates an as clean as possible mucosa for the introduction of a new, healthy microbiota.

In alternative embodiments, methods and products of manufacture as provided herein deliver an ultrasonic or acoustic pressure wave energy to an in situ biofilm location, e.g., delivered to a body cavity (e.g., a colon) in need of treatment, by e.g., the use of an acoustic waveguide. An acoustic waveguide can transmit acoustic waves in frequencies ranging from the audible to the ultrasonic spectrum. An acoustic waveguide can transmit acoustic waves via a flexible wire or rod to a distal location where the outputted acoustic energy can induce acoustic streaming, stable cavitation and/or inertial cavitation in a liquid.

In alternative embodiments, ultrasonic waveguides used to practice or make products of manufacture and methods as provided herein can use ultrasonic waveguides or components as described in for example: WO1989006515A1, applying ultrasonic waveguide technology to ultrasonic angioplasty; U.S. Pat. Nos. 7,335,180 and 6,617,760, describing ultrasonic resonators, or U.S. patent application publication 2015/015057 1A1. In alternative embodiments, ultrasonic waveguides used to practice or make products of manufacture and methods as provided herein have no diameter size requirements, and an ultrasonic waveguide of larger diameter can be used to transmit acoustic waves in a body cavity such as the colon, thus, a higher power output can be released in a liquid or a fluid medium. The ultrasonic waveguide can have a diameter from between about 0.1 mm to a 30 mm, or between about 0.5 mm and 20 mm. The ultrasonic waveguide can be a cylindrical wire or rod, a hollow tube, a conical wire or rod or tube or a rectangular bar of various thicknesses, e.g., having a thickness of between bout 0.1 mm to a 30 mm, or between about 0.5 mm and 20 mm.

In alternative embodiments, the acoustic waveguide is of a uniform thickness and cross-sectional area from its proximal end attached to the transducer, to its distal tip. In another embodiment, the waveguide is of a variable thickness and cross-sectional area starting with a larger cross-sectional area at its proximal end attached to the transducer and progressively getting thinner towards its distal end. By tapering the cross-sectional area, the vibration amplitude of the acoustic wave transmitted via the acoustic waveguide is amplified and results in a larger amplitude displacement at the tip, resulting in a more powerful emitted acoustic field. An acoustic waveguide can have two or more tapered sections to enable amplification of the vibratory displacement of the tip.

In alternative embodiments, the acoustic waveguide is a tapered ultrasonic transmission guide wire, which can be made of a super-elastic metal alloy, for example, as described in U.S. Pat. No. 6,450,975. In alternative embodiments, the tip at the end of the acoustic waveguide has a diameter between about 0.5 mm and 30 mm. The shape of the tip can have different geometries, for example, it can have a semi-spherical tip, a spherical tip, a cylinder tip, a square tip, a pear-shaped tip or a flat tip. In alternative embodiments, the length of the acoustic waveguide is between about 1 cm to 3500 cm or more, for example, the acoustic waveguide can have a length to reach an inner location like the rectum or a more distal body cavity location, e.g., like the cecum, when treating a gastrointestinal tract, e.g., a colon; thus, therapy can be applied throughout the length of the large intestine. In alternative embodiments, the length of the acoustic waveguide is made to respond to the resonance of the imparted frequency as to allow the distant end of the acoustic waveguide to vibrate at a larger amplitude, therefore releasing more acoustic energy at the location being treated.

The amplitude of the acoustic waveguide tip also depends on the power that is transmitted through it. In alternative embodiments, the displacement amplitude of the tip varies from between about 1 μm to 1000 μm peak-to-peak. A maximum pressure is produced in the immediate vicinity of the acoustic waveguide tip, with a rapid decrease in pressure with increasing distance from the acoustic waveguide tip. The power transmitted via the acoustic waveguide is to be enough to produce stable cavitation, inertial cavitation and microstreaming in the immediate surroundings around the tip, so when the acoustic waveguide is maneuvered close to the tissue, e.g., within about 2 mm to 30 mm, the acoustic pressure impacting the biofilm is reduced so there is minimal injury caused to the mucosa and epithelium.

In another embodiment, the amplitude of the waveguide tip is increased if the diameter of the metal waveguide is relatively smaller than the catheter into which it has been inserted into; this may occur because, in addition to the longitudinal wave imparted by the transducer to the waveguide, a flexular and transverse wave mode is introduced which may increase the displacement amplitude at the tip. Although this action may not be wanted during ultrasonic angioplasty as it could cause injury to the surrounding arterial wall, it may be of benefit during biofilm treatment in the bowel as there is much more room for the acoustic waveguide to oscillate and impart its energy.

In alternative embodiments, the acoustic waveguide is made out of or manufactured using a metal that can transmit sonic and ultrasonic waves, for example, using metals such as aluminium and its alloys, titanium and its alloys, nickel titanium (nitinol) and stainless steel and its alloys, or any other appropriate metal.

In alternative embodiments, the acoustic waveguide is inserted into a working channel of a product of manufacture (e.g., an endoscope) or overtube as provided herein. Insertion of the product of manufacture or overtube within a catheter is done to prevent damage to the product of manufacture or overtube. The catheter can be made of plastic, nylon, a polymer, flexible metal and other materials known to those in the art.

In alternative embodiments, the transducer transmitting the acoustic energy in the waveguide is situated parallel to the acoustic waveguide to transmit longitudinal waves, or it can be attached to the waveguide perpendicularly to transmit a shear wave to the waveguide. In another embodiment, both types of transducers are combined to transmit a combination of a longitudinal and shear waves in the waveguide.

In another embodiment, the waveguide tip is covered in protrusions, e.g., soft protrusions such as bristles, to enhance the hydrodynamic action of the tip and the mixing of the nano- or micro-bubbles in the vicinity of the tissue to enhance the biofilm removal action; the vibration of the bristles can enable acoustic energy transfer. The energy transfer to the biofilm is thought to depend on the frequency and amplitude applied. Thus, in alternative embodiments, products of manufacture as provided herein can form or generate oscillatory fluid motions and pressure waves to generate additional shear forces on the biofilm matrix and bacteria residing therein. The oscillation of entrapped air bubbles also can enhance shear forces by micro-streaming and cavitation effects.

In alternative embodiments, the protrusions are made from material comprising a polyamide like nylon, a polymer like polyester or silicone, a polyethelene, polytetrafluoroethylene (PTFE) (TEFLON™), polyacrylonitrile acrylic fibers, elastomeric materials, and combinations thereof. The protrusions can have smooth rounded ends to prevent or minimise abrasion of the mucosal surface.

In another embodiment, the location of the transducer driving the acoustic waveguide is placed near the tip of the endoscope to minimise the acoustic wave's loss due to the distance travelled. The short length acoustic waveguide can be attached to the transducer close to the product of manufacture's (e.g., endoscope's) tip prior to insertion of the product of manufacture in the body, e.g., into the body cavity to be treated, e.g., into a colon. In this embodiment the acoustic waveguide can have a length of between about 0.1 cm to about 12 cm, or between about 1.0 cm and 10 cm.

In another embodiment, the tip of the product of manufacture (e.g., endoscope) houses a motor that vibrates at between 10 Hz to about 10,000 Hz. The motor can cause the waveguide to vibrate in an oscillating motion so its tip looks like it is moving in an arc. The waveguide can be moved between 1 and 20 degrees from the neutral position during each oscillation. The vibrating motor can be a DC motor, an AC motor, an electromagnetic motor or any other motor or driver that can impart an oscillating motion to the attached acoustic waveguide.

In one embodiment, the acoustic waveguide is attached to the vibrating motor and the product of manufacture (e.g., endoscope) is inserted in the body cavity to be treated. In this embodiment the waveguide does not vibrate ultrasonically; however, the rapid oscillating movement of the tip is still enough to cause biofilm degradation and/or dispersal. In this embodiment, the acoustic waveguide may have a length of between about 1.0 cm and 10 cm, or 0.1 cm and 12 cm, or 0.5 cm and 8 cm. The acoustic waveguide material in this embodiment can comprise a metal, plastic, polyamide, elastomer, polymer or other materials, or a combination of materials. The waveguide tip at the end of the acoustic waveguide can have a diameter of between about 0.5 mm and 30 mm, or 0.1 mm and 60 mm.

In one embodiment, to enhance the brushing and biofilm removing effect, the acoustic waveguide and/or its tip are manufactured or configured as described, e.g., in U.S. Pat. No. 8,046,861, which describes a power toothbrush using acoustic wave action for cleansing of teeth. The shape of the tip can have various different geometries, for example, the shape of the tip can be a semi spherical tip, spherical tip, a cylinder tip, a square tip, a pear-shaped tip or a flat tip.

In one embodiment, a device as described e.g., in WO 2016/049472, is used to impart vibration to products of manufacture as provided herein to reduce disrupt or breakup biofilm; and this device can comprise a vibration tip, and optionally the vibration tip is sized and shaped to couple to a vibrator and to receive a vibration therefrom, wherein the vibration tip is sized and shaped to conduct the vibration from the vibrator to the product of manufacture. For this embodiment, an operator can place the product of manufacture's vibrating waveguide near the tissue to be treated to degrade and/or dislodge the biofilm by exploiting the hydrodynamic effect of the vibrations. In one embodiment, vibrating and ultrasonic probes coexist and be used serially or simultaneously in any product of manufacture as provided herein.

In alternative embodiments, the term “ultrasound emitter(s)” as used herein refers to different types of ultrasound emitting elements such as ring transducers, disc transducers, piezoelectric transducers, film transducers, micromachined ultrasound transducers, capacitive micromachined ultrasound transducers, piezoelectric micromachined ultrasound transducers, transducers arranged in arrays such as linear/convex/phased/radial ultrasound arrays and any type of device or element that can transmit an ultrasound frequency or a range of frequencies.

In alternative embodiments, the term “tissue” as used herein refers to different tissue types, such as mucosa, e.g., mucosa as found in the GI tract, bowel, bladder and pleura as found in the lungs.

In alternative embodiments, the term “biofilm” as used herein refers to a group of microorganisms that are encased in an extracellular matrix composed of extracellular polymeric substances.

In alternative embodiments, the term “endoscope” as used herein refers to a colonoscope, cystoscope, cystoscopenephroscope, bronchoscope, enteroscope and laparoscope.

In alternative embodiments, the term “active area” as used herein refers to the area of a product of manufacture (e.g., endoscope) as provided herein that has ultrasound emitters placed on it which emit an ultrasound frequency acoustic wave.

In alternative embodiments, the term “removal of the biofilm” as used herein refers to the removal of an attached or adherent biofilm from a tissue site, including complete or 100% removal, or substantial removal, which can be removal of between about 80% and 99.5%, or between about 85% and 99%, of the attached or adherent biofilm from a tissue site, e.g., from a mucosal surface.

In alternative embodiments, the term “degradation of the biofilm” as used herein refers to the removal of smaller sections of the biofilm from the tissue site, or it can refer to any amount of breaking up of a biofilm, or causing any part of a biofilm to be non-adherent to a tissue, e.g., a mucosa.

In alternative embodiments, provided is a product of manufacture, and a method, comprising ultrasound generator and an ultrasound emitter or plurality of emitters, for use as an ultrasound endoscope operating in a radial and/or longitudinal mode. In one embodiment, an ultrasound endoscope is placed in a liquid or fluid-filled body cavity, e.g., a colon, in close distance (e.g., between about 0.5 to 10 cm) from a target tissue, e.g., a colon mucosa. In alternative embodiments, the ultrasound generator can produce an electrical signal that drives an ultrasound emitter or plurality of emitters to produce radial longitudinal and/or omnidirectional ultrasound waves along the length of the ultrasound endoscope (e.g., spaced along the distal 50% to 99% of the length of the endoscope) and/or its tip or end. The ultrasound waves so generated can cause non-inertial cavitation, inertial cavitation and/or microstreaming in the liquid or fluid surrounding the ultrasound endoscope, thus causing remove, disruption or degrading of the biofilm (e.g., mucosal adherent biofilm) from the tissue. In alternative embodiments, the fluid or liquid surrounding the ultrasound endoscope, or the end of the endoscope, is a water (e.g., ozone water, distilled water, activated water, or electrolyzed water) or saline, and the fluid or liquid can be delivered by the endoscope or an ancillary device or attachment to the endoscope. In another embodiment, the water is mixed with a biocide e.g., ozone, to increase the biocidal effect against the biofilm and the bacteria within. In alternative embodiments, biocides that are used or administered comprise: activated water, electrolyzed water, sodium hypochlorite, formalin, gluteraldehyde, boric acid, antiseptics or antibiotics and/or mixtures thereof, e.g., as described herein. In another embodiments, other compositions can be added to the administered fluids or liquid, e.g., enzymes such as DNAase and/or surfactants such as soaps.

Methods as provided herein also comprise a pre-treatment (before ultrasound treatment) of the biofilm, e.g., pre-treatment of bacteria in the biofilm (i.e., the infection of the biofilm) by utilising an antibiotic or antibiotics, or anti-biofilm agents in combination with antibiotics, before the use of products of manufacture as provided herein for an ultrasound treatment. This length of the pre-treatment period can be between about one day to two (2) weeks, and can last as long as several or many weeks, e.g., can last for between about 2 to 20 weeks. Antibiotics or anti-biofilm supplement or supplements can continue to be given (e.g., given orally, intramuscularly, intravenously) during and/or after a treatment with a product of manufacture as provided herein. For example, an anti-biofilm supplement or supplements are given orally to the patient, e.g., in an enterically coated capsule or tablet so the active ingredients of the capsule are released in the small intestine and large intestine. The anti-biofilm supplements, e.g., supplements given during the pre-treatment and/or post-treatment period, can be one or any combination of the following: N-Acetyl Cysteine, bismuth and bismuth analogues, alginate and alginate analogues, catechins and epicatechins like epigallocatechin gallate, ethylene-diaminetetraacetic acid (EDTA), alpha lipoic acid, mesalazine, sulfasalazine and their analogues and other agents known in the art to have an anti-biofilm effect, including those as described herein. The antibiotics can be selected any of the following: tetracycline, doxycycline, minocycline, penicillin and its derivatives for example amoxicillin, metronidazole, carbapenem and its derivatives, gentamicin and other antibiotics and combinations thereof. In alternative embodiments, the ultrasound emitter can be attached to or line the outside layer of the endoscope may operate in frequencies between 10 Khz to 30 Mhz.

In alternative embodiments, methods as provided herein also comprise administration of fluids or liquids with nano- or micro-bubbles, or comprise generation of nano- or micro-bubbles in the vicinity of the tissue being treated or cleansed or debrided of biofilm. In alternative embodiments, ultrasound frequency and/or amplitude is either static or modulated during treatment, e.g., throughout the treatment, to initiate and/or maximise non-inertial cavitation, inertial cavitation and microstreaming of the bubbles present in the liquid or fluid medium.

In alternative embodiments, ultrasonically activated sections of a product of manufacture (e.g., endoscope) as provided herein is covered with a plurality of ultrasound emitters emitting ultrasound in a radial and/or longitudinal mode. The ultrasound emitter or emitters can be: flexible (for example, film transducers); non-flexible (for example, disc transducers); or, any other type of ultrasound emitter that can be used or combined to suit the specific design of a product of manufacture (e.g., endoscope) as provided herein.

In another embodiment, hollow ring transducers line the ‘active’ area of the product of manufacture (e.g., endoscope), and these transducers can emit ultrasound in a longitudinal and/or radial mode. In alternative embodiments, the transducers are placed in close proximity with each other (e.g., about 1 to 5 cm apart), or they can be spaced further apart (e.g., about 5 to 20 cm) to allow for the bending of the product of manufacture.

In alternative embodiments, a base fluid or liquid used to transfer the ultrasound wave to the tissue from the ultrasound endoscope can be any form of water (e.g., ozone water, distilled water, activated water, or electrolyzed water), or saline, or an oil, or a combination thereof. In another embodiment, the base fluid or liquid is administered using a product of manufacture as provided herein, or an ancillary device, and the administered base fluid or liquid can comprise a biocide or an antibiotic. In alternative embodiments, biocide comprises ozone, e.g., in concentrations of between about 0.1 ppm to 10 ppm, or between 1.0 ppm to 7 ppm.

In alternative embodiments, the biocide and/or antibiotic is used to neutralize (e.g., kill) any bacteria freed from the biofilm or in a disrupted or degraded biofilm (e.g., in a biofilm fragment no longer adherent to a mucosa) so the bacteria cannot settle to another part of the body (e.g., another section of the colon) and set up a new biofilm cluster.

In alternative embodiments, a product of manufacture (e.g., endoscope) as provided herein comprises or comprises use of a suction channel, or uses a suction ancillary device, to remove the liquid medium after each round of treatment, e.g., after each round of biofilm disruption or degrading by application of ultrasound treatment.

In alternative embodiments, a product of manufacture (e.g., endoscope) as provided herein comprises or comprises use of a temperature gauge, e.g., a temperature gauge embedded in the product of manufacture, e.g., a temperature gauge embedded every few centimetres (e.g., every 3 to 30 cm) of the ‘active’ ultrasound section to monitor the surrounding fluid or liquid medium temperature in order to prevent thermal damage to the tissue.

In alternative embodiments, the ultrasound emitter or emitters are covered by a sheath, a sleeve, an overtube or equivalent on the outside of the product of manufacture (e.g., endoscope) to prevent contact between the ultrasound emitters and the tissue. The sheath, overtube or sleeve can be made of an ultrasound transparent material to allow (e.g., substantially all of) the acoustic wave to travel through it. In other embodiments, the ultrasonic emitters are exposed and in contact with the liquid medium.

In alternative embodiments, the ultrasound emitter or emitters continuously cover the entire ‘active’ area of the product of manufacture (e.g., endoscope) or they cover segments of the product of manufacture. In alternative embodiments, the ‘active’ area of the product of manufacture is a flexible tube to allow insertion and manoeuvring within the body, e.g., within a colon.

In alternative embodiments, a product of manufacture (e.g., endoscope) as provided herein comprises or comprises use of a camera, which can be built into or (optionally removably) attached to the product of manufacture to allow the operator to guide the product of manufacture to the area to be treated. In alternative embodiments, the product of manufacture can be manually maneuvered (e.g., guided by an image produced by the camera) around the body cavity being treated in order to move it closer to the mucosa if need be in order to ensure the ultrasound energy is effectively reaching the area and stable/non-inertial cavitation, inertial cavitation and/or microstreaming is dissolving the biofilm.

In alternative embodiments, microbubbles or nanobubbles are introduced into the area treated via external means or created in vivo via a cavitation effect. In alternative embodiments, externally produced bubbles are a predetermined size and can be matched to the frequency of the ultrasound emitters to cause a stable/non-inertial cavitation, inertial cavitation and/or microstreaming effect in vivo. Externally produced bubbles (e.g., including commercially available microbubbles such as OPTISON™, LEVOVIST™ and/or SONOVUE™; or microbubbles made using a machine, e.g., a microbubble generator, before delivering the liquid in vivo) may be stabilized by using surfactants, lipids, polymers etc to cause a stable/non-inertial cavitation, inertial cavitation and/or microstreaming effect in vivo.

In alternative embodiments, the power transmitted to the tissue should be at a level that causes little or minimal damage while still effectively treating or disrupting the biofilm covering or adherent to the tissue, e.g., the mucosa. In alternative embodiments, the power transmitted to the tissue can be between about 0.01 watt per square centimetre to about 300 watts per square centimeter, or can be between about 0.1 and 200 watts per square centimeter. In alternative embodiments, the transmitted ultrasound is delivered in a pulsed or a continuous mode.

In alternative embodiments, bodily parts treated by methods as provided herein, or by products of manufacture as provided herein, include the large intestine, small intestine, stomas, bile and pancreatic ducts, mouth, ears, trachea, oesophagus, stomach, urinary bladder, sinuses, bronchi, bronchioles and lungs; additionally, under some circumstances, cerebrospinal fluid-filled cavities of the central nervous system may be treated, and modified probes can be used in arteries and veins.

In alternative embodiments, the liquid or fluid medium delivered to the body space or cavity to be treated is at approximately body temperature, or can be between about 1 to 38 degrees C. The temperature of the liquid or fluid medium delivered to the body space or cavity to be treated can be lower than body temperature to allow a few minutes of ultrasound treatment before the fluid's or liquid's temperature is elevated to a degree that it necessitates the stopping of treatment. In alternative embodiments, the liquid or fluid medium in the area of treatment in situ is kept low enough to prevent thermal damage to any tissue.

In another embodiment, spacing rings are placed every few centimetres (e.g., every about 5 to 20 cm) of the ultrasonically activated product of manufacture (e.g., endoscope) area to prevent contact of the ultrasound emitting area with the tissue; this allows the liquid medium to interface between the transducer and tissue being treated and ensures stable and/or non-inertial cavitation, inertial cavitation and micro-streaming is enabled throughout the ultrasonically activated area. In alternative embodiments, the spacing rings extend out from between 3 to 20 cm.

In alternative embodiments, methods as provided herein use a balloon or series (plurality) of balloons to treat segments of a body part or cavity, e.g., a large intestine or colon, so as to ensure the liquid or fluid medium has filled the particular segment to ensure effective transmission of the ultrasound waves to the tissue and the target biofilm. In alternative embodiments, the balloon or plurality of balloons are placed using an attachment to a product of manufacture (e.g., endoscope) as provided herein, or can be placed using an ancillary device. Treating smaller sections of a body cavity (e.g., the GI tract, or the colon) at one time will ensure no air pockets are created along the walls of the body cavity, and as such the applied ultrasound energy effectively induces stable/non inertial cavitation, inertial cavitation and microstreaming against the tissue surface.

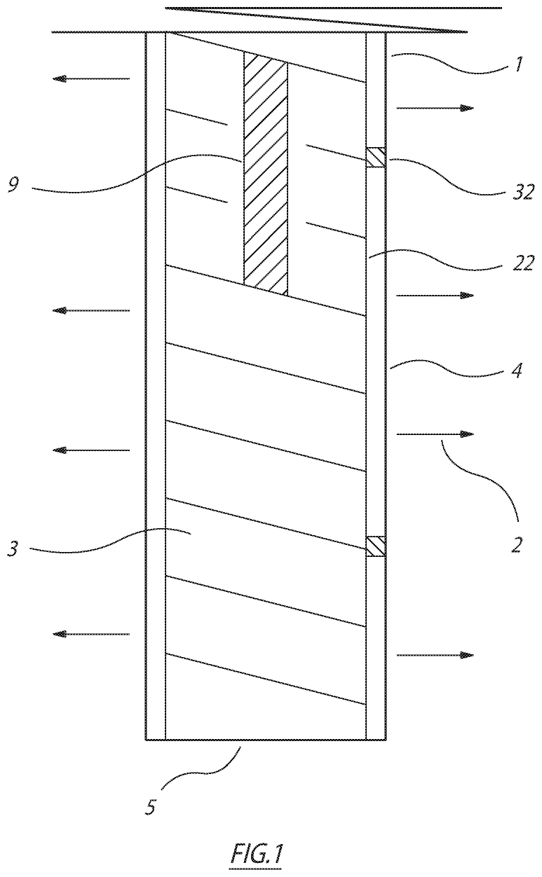

FIG. 1 schematically illustrates an exemplary product of manufacture as provided herein, comprising: an ultrasound endoscope with a one piece and/or very few pieces of flexible ultrasound emitter(s) (3) diagonally wrapped around the endoscope's body (1). A cross sectional area shows an electrode cable (9) running from the ultrasound generator to the distal tip of the ultrasound endoscope (5) powering the flexible ultrasound emitter(s) (3). The ultrasound wave (2) travels perpendicularly and/or radially away from the axis of the endoscope through the liquid medium surrounding the ultrasound endoscope to the tissue where it exerts its effect. Temperature sensors (32) are spaced along the length of the ultrasound endoscope (1) to monitor the temperature of the surrounding liquid as to allow cessation of treatment once the temperature of the surrounding liquid medium is increased above a predetermined level. The temperature sensors (32) are housed between the inner endoscope body (22) and the endoscope sheath (4) which is of a material which does not attenuate or alter the frequency of the ultrasound wave emitted by the flexible ultrasound emitter(s) (3). The temperature sensor (32) can be placed on the outside surface of the sheath (4) as long as it sits flat against it and does not protrude, or minimally protrudes, from the sheath surface (for example, only protrudes from the outer body or sheath of the exemplary product of manufacture by between about 0.5 or 1 mm to 2 or 3 cm).

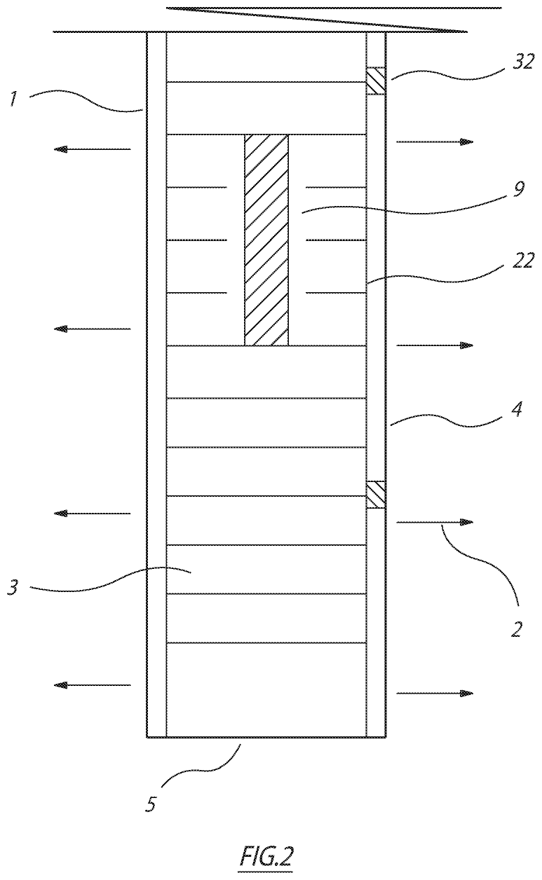

FIG. 2 schematically illustrates an exemplary ultrasound endoscope as provided herein comprising: multiple flexible ultrasound emitters (3) horizontally wrapped around the endoscope's body (1). A cross sectional area shows an electrode cable (9) running from the ultrasound generator to the distal tip of the ultrasound endoscope (5) powering the flexible ultrasound emitters (3). The ultrasound wave (2) travels perpendicularly and/or radially away from the axis of the endoscope through the liquid medium surrounding the ultrasound endoscope to the tissue where it exerts its effect. Temperature sensors (32) are spaced along the length of the ultrasound endoscope (1) to monitor the temperature of the surrounding liquid as to allow cessation of treatment once the temperature of the surrounding liquid medium is increased above a predetermined level. The temperature sensors (32) are housed between the inner endoscope body (22) and the endoscope sheath (4) which is of a material which does not attenuate or alter the frequency of the ultrasound wave emitted by the flexible ultrasound emitters (3). The temperature sensor (32) can be placed on the outside surface of the sheath (4) as long as it sits flat against it and does not protrude, or minimally protrudes, from the sheath surface.

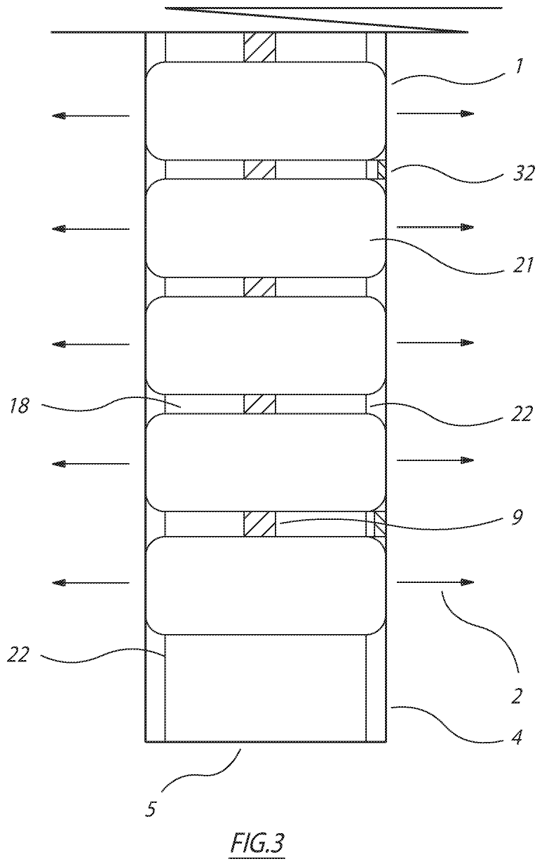

FIG. 3 schematically illustrates an exemplary ultrasound endoscope with ring-shaped transducers placed inside the endoscope's sheath or outer body at regular intervals (for example, placed every 3 to 10 or 20 cm). An electrode cable (9) running from the ultrasound generator to the distal tip of the ultrasound endoscope (5) powering the ring-shaped ultrasound transducers (21). The ultrasound wave (2) travels perpendicularly and/or radially away from the axis of the endoscope through the liquid medium surrounding the ultrasound endoscope to the tissue where it exerts its effect. Temperature sensors (32) are spaced along the length of the ultrasound endoscope (1) to monitor the temperature of the surrounding liquid as to allow cessation of treatment once the temperature of the surrounding liquid medium is increased above a predetermined level. The temperature sensors (32) are housed between the inner endoscope body (22) and the endoscope sheath (4) which is of a material which does not attenuate or alter the frequency of the ultrasound wave emitted by the ring-shaped ultrasound transducers (3). The temperature sensor (32) can be placed on the outside surface of the sheath (4) as long as it sits flat against it and does not protrude, or minimally protrudes, from the sheath surface. The ring-shaped transducers are separated by a gap (18) to allow the ultrasound endoscope to be maneuvered around the body cavity it has been inserted into.

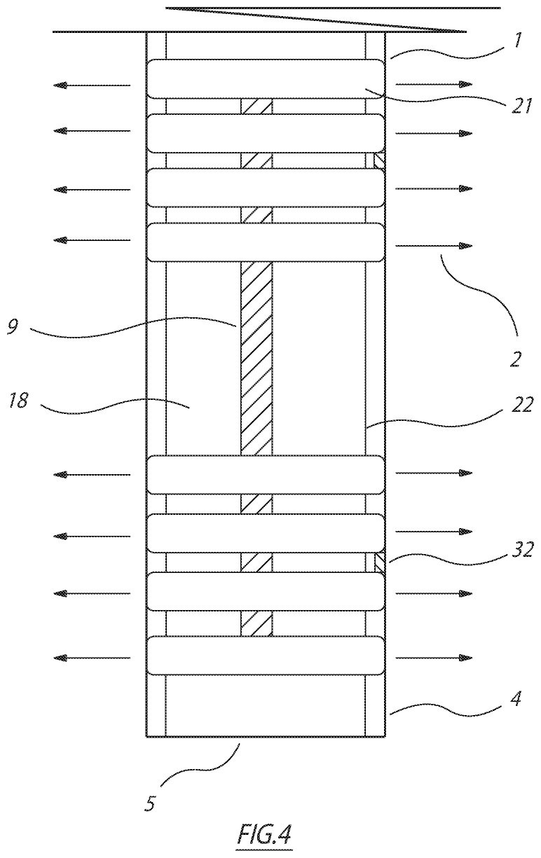

FIG. 4 schematically illustrates an exemplary ultrasound endoscope with thinner ring transducers (21) placed inside the endoscope's sheath (4) with larger gaps between segments of ring transducers. An electrode cable (9) running from the ultrasound generator to the distal tip of the ultrasound endoscope (5) powering the ring-shaped transducers (21). The ultrasound wave (2) travels perpendicularly and/or radially away from the axis of the endoscope through the liquid medium surrounding the ultrasound endoscope to the tissue where it exerts its effect. Temperature sensors (32) are spaced along the length of the ultrasound endoscope (1) to monitor the temperature of the surrounding liquid as to allow cessation of treatment once the temperature of the surrounding liquid medium is increased above a predetermined level. The temperature sensors (32) are housed between the inner endoscope body (22) and the endoscope sheath (4) which is of a material which does not attenuate or alter the frequency of the ultrasound wave emitted by the ring-shaped transducers (3). The temperature sensor (32) can be placed on the outside surface of the sheath (4) as long as it sits flat against it and does not protrude, or minimally protrudes, from the sheath surface. The ring-shaped transducers are separated by a gap (18) to allow the ultrasound endoscope to be maneuvered around the body cavity it has been inserted into.

FIG. 5 schematically illustrates an exemplary wide beam ultrasound emitter array endoscope attachment in the closed position. This attachment can be used on its own attached to a normal endoscope or in combination with an ultrasound endoscope. This attachment can also be a built-in component of an ultrasound or non-ultrasound endoscope. It comprises of a base plate (6) which rests on the endoscope body (1). The apparatus can be clipped on to the endoscope body (1) by the wide beam assembly attachment (29). An array of wide beam ultrasound emitters (8) sits on top of the attachment. The wide beam arrays transmit the ultrasound wave (2) towards the tissue. The wide beam ultrasound array attachment mechanism has a curvilinear shape (10) to allow easy insertion and extrusion from the body. The wide beam ultrasound array can be attached anywhere along the body of the endoscope (1) but a gap needs to be left proximally to its tip (5) to allow for the manoeuvring of the endoscope. An electrode cable (9) runs from the ultrasound generator to the wide beam ultrasound attachment powering the ultrasound arrays. The wide beam ultrasound array can transmit ultrasound energy in a continuous or pulsed mode.

FIG. 6 schematically illustrates an exemplary wide beam ultrasound emitter array endoscope attachment described in FIG. 5 in the open position. This attachment can be used on its own attached to a normal endoscope or in combination with an ultrasound endoscope. This attachment can also be a built-in component of an ultrasound or non-ultrasound endoscope. It comprises of a base plate (6) which rests on the endoscope body (1). The apparatus can be clipped on to the endoscope body (1) by the wide beam assembly attachment (29). An array of wide beam ultrasound emitters (8) sits on top of the attachment. The wide beam arrays transmit the ultrasound wave (2) towards the tissue. The wide beam ultrasound array attachment mechanism has a curvilinear shape (10) to allow easy insertion and extrusion from the body. The wide beam ultrasound array can be attached anywhere along the body of the endoscope (1) but a gap needs to be left proximally to its tip (5) to allow for the manoeuvring of the endoscope. An electrode cable (9) runs from the ultrasound generator to the wide beam ultrasound attachment powering the ultrasound arrays. The wide beam ultrasound array can transmit ultrasound energy in a continuous or pulsed mode. The wide beam ultrasound array attachment has a lifting mechanism (7) which can lift the ultrasound attachment anywhere between 1 to 90 degrees. The ultrasound attachment is kept in the closed position via an anchoring mechanism (33) which is situated on the base plate (6).

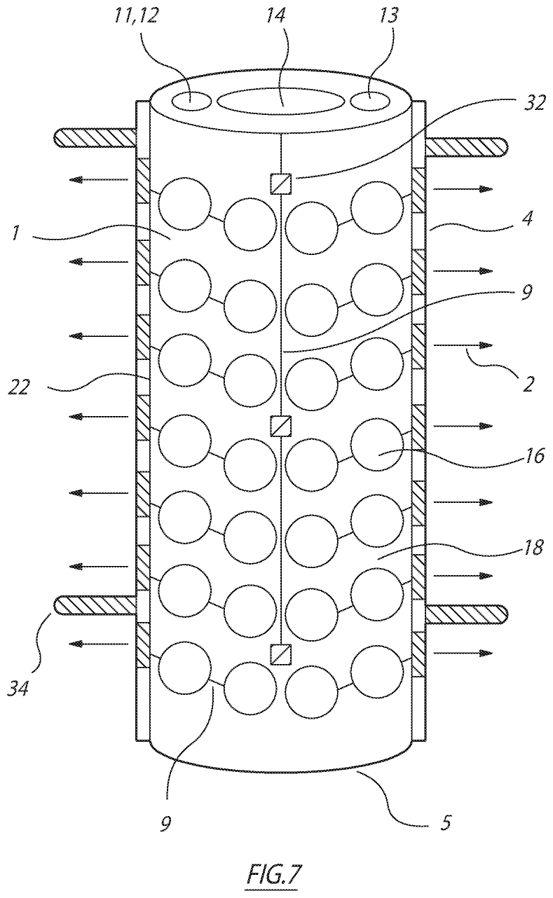

FIG. 7 schematically illustrates an exemplary ultrasound endoscope with an irrigation (11) and/or suction (12) channel, a working channel (14), a camera (13) and a plurality of disk-shaped ultrasound emitters placed inside the endoscope's sheath at regular intervals horizontally and longitudinally along the endoscope body (1) up to nearly the endoscope's tip (5). An electrode cable (9) running from the ultrasound generator to the multiple ultrasound emitters (16) of the ultrasound endoscope (1) powering them. The ultrasound wave (2) travels perpendicularly and/or radially away from the axis of the endoscope through the liquid medium (20) surrounding the ultrasound endoscope to the tissue where it exerts its effect. Temperature sensors (32), powered by an electrode cable (9), are spaced along the length of the ultrasound endoscope (1) to monitor the temperature of the surrounding liquid as to allow cessation of treatment once the temperature of the surrounding liquid medium is increased above a predetermined level. The temperature sensors (32) are housed between the inner endoscope body (22) and the endoscope sheath (4) which is of a material which does not attenuate or alter the frequency of the ultrasound wave emitted by the ultrasound emitters (16). The temperature sensor (32) can be placed on the outside surface of the sheath (4) as long as it sits flat against it and does not protrude, or minimally protrudes, from the sheath surface. The ultrasound emitters (16) are separated by a gap (18) to allow the ultrasound endoscope to maintain its flexibility so it can be maneuvered around the body cavity it has been inserted into. Flexible spacing rings (34) are placed at regular intervals to prevent the endoscope from resting against the tissue being treated which would interfere with the treatment as there needs to be a liquid interface between the ultrasound endoscope and the tissue in order for the biofilm degradation and/or removal to be effective.

FIG. 8 schematically illustrates an overview of how an exemplary ultrasound endoscope operates when the ultrasound emitters are activated. The ultrasound endoscope (1) is inserted into the body cavity to be treated. The endoscope tip (5) is placed past the border of the area to be treated to allow the ultrasonically active area of the endoscope to fully cover the tissue (19) to be treated. The ultrasound endoscope has temperature sensors (32) along the length of the endoscope body (1) to monitor the temperature of the liquid medium (20). The temperature sensors (32) are housed between the inner endoscope body (22) and the endoscope sheath (4) which is of a material which does not attenuate or alter the frequency of the ultrasound wave (2) emitted by the ultrasound emitters. When the ultrasound signal is activated and transmitted to the ultrasound emitters they emit a radial and/or longitudinal wave (2) and it is transmitted to the tissue (19). The ultrasound wave causes the liquid medium's microbubbles and/or nanobubbles to undergo non inertial cavitation (25), inertial cavitation (26) and microstreaming (27) which results in the degradation and eventual removal of biofilm (24) from the tissue (19). The liquid medium (20) can have a biocide such as ozone added to it to neutralize any microorganisms released from the biofilm (24). The ultrasound endoscope can be maneuvered around the body cavity by the physician and in close approximation to the tissue (19) to achieve effective degradation and removal of the biofilm (24).

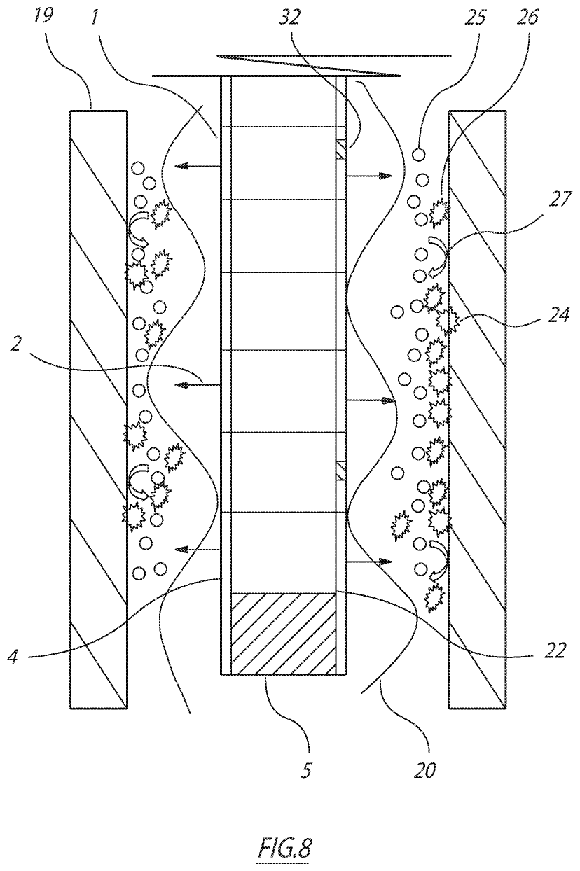

FIG. 9 schematically illustrates an ultrasound emitter array endoscope attachment, attached past the distal end of an endoscope. This attachment can be used on its own attached to a normal endoscope or in combination with an ultrasound endoscope. It comprises an anchoring mechanism (33) which rests on the endoscope body (1) and near the endoscope tip (5). An array of radial ultrasound emitters, in this example ring transducers (21) sit on top of the attachment. An electrode cable (9) runs from the ultrasound generator to the ultrasound attachment powering the ultrasound emitters. Temperature sensors (32) are spaced along the length of the attachment to monitor the temperature of the surrounding liquid as to allow cessation of treatment once the temperature of the surrounding liquid medium is increased above a predetermined level. Flexible spacing rings (34) are placed at regular intervals to prevent the endoscope from resting against the tissue being treated which would interfere with the treatment as there needs to be a liquid interface between the ultrasound endoscope and the tissue in order for the biofilm degradation and/or removal to be effective. The attachment tip (17) is rounded to allow insertion in the body. The attachment can measure from 0.5 cm to 20 cm in length.

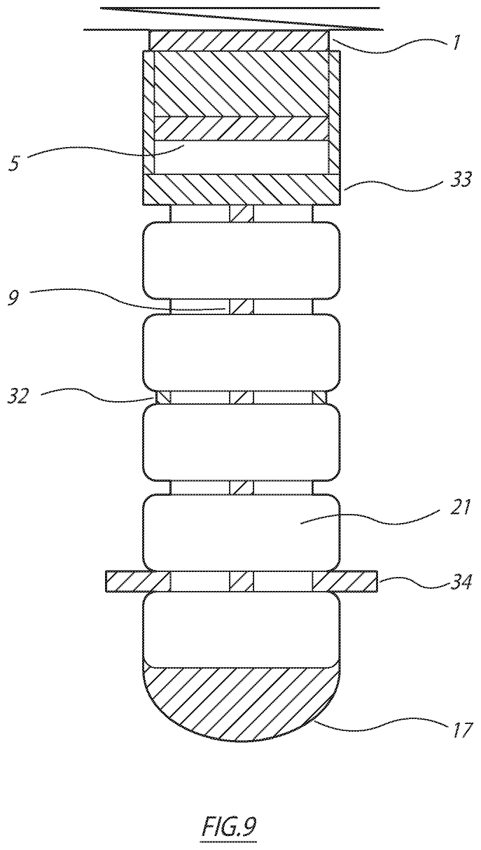

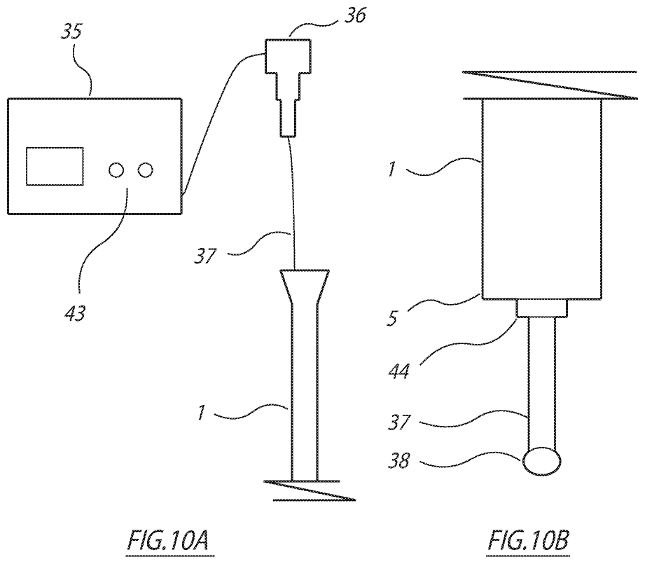

FIG. 10a schematically illustrates an exemplary ultrasonic waveguide (37) that is powered by an external ultrasound generator (35). The ultrasonic generator's power output, duty cycle and other functions are controlled via the unit's controls (43). The ultrasonic generator is connected to and runs an ultrasonic horn (36). The ultrasonic horn has a waveguide (37) connected at its tip as to transfer the ultrasonic energy to the location to be treated via the endoscope (1).

FIG. 10b schematically illustrates a close up of the distal part of the exemplary endoscope (1), and the image shows how the ultrasonic waveguide (37) when situated inside a catheter (44) and inserted into the endoscope's working channel is extended past the endoscope tip (5) in order to vibrate and transfer its energy and acoustic waves to the surrounding liquid, which transmits the acoustic waves to the tissue to be treated. The ultrasonic waveguide has a tip (38) attached to its distal end in order to enhance and increase the transmission of the acoustic energy to the surrounding liquid. The endoscope can be maneuvered close to the tissue that is being treated as to maximise the amount of energy output of the waveguide reaching the tissue being treated.

FIG, 11a schematically illustrates an exemplary ultrasonic waveguide (37) and its tip (38), that is powered by an external ultrasound generator (35); however, in this embodiment the waveguide and ultrasound transducer are located inside the endoscope (1). The ultrasonic generator's power output, duty cycle and other functions are controlled via the unit's controls (43). The ultrasonic generator is connected to and runs the ultrasound transducer located in the endoscope via a cable (9) that runs inside the endoscope (1).

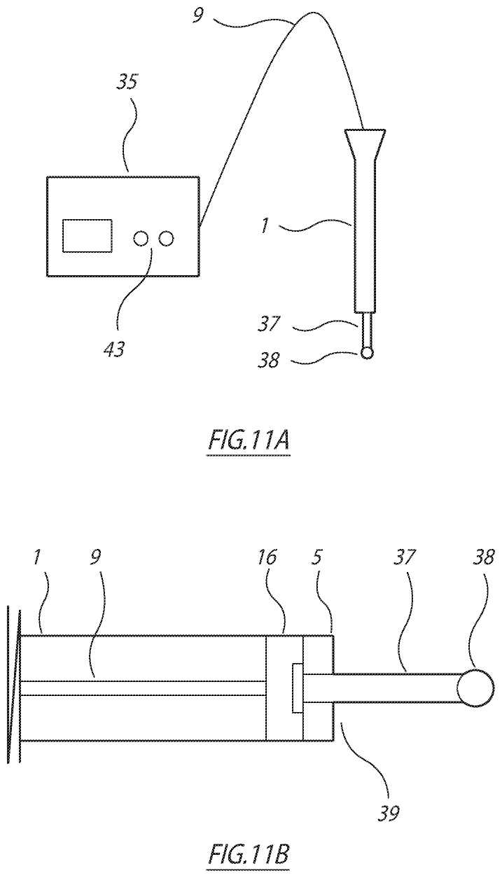

FIG. 11b schematically illustrates a close up of the distal part of the exemplary endoscope (1), and it is illustrated where the ultrasonic waveguide (37) with a rounded tip (38) connected via an attachment mechanism (39) to the ultrasound transducer (16) which is connected to the external ultrasound generator via a cable (9), extends past the endoscope tip (5) in order to vibrate and transfer its energy and acoustic waves to the surrounding liquid which transmits the acoustic waves to the tissue to be treated. The ultrasonic waveguide has a rounded tip (38) attached to its distal end in order to enhance and increase the transmission of the acoustic energy to the surrounding liquid. The endoscope can be maneuvered close to the tissue that is being treated as to maximise the amount of energy output of the waveguide reaching the tissue being treated.

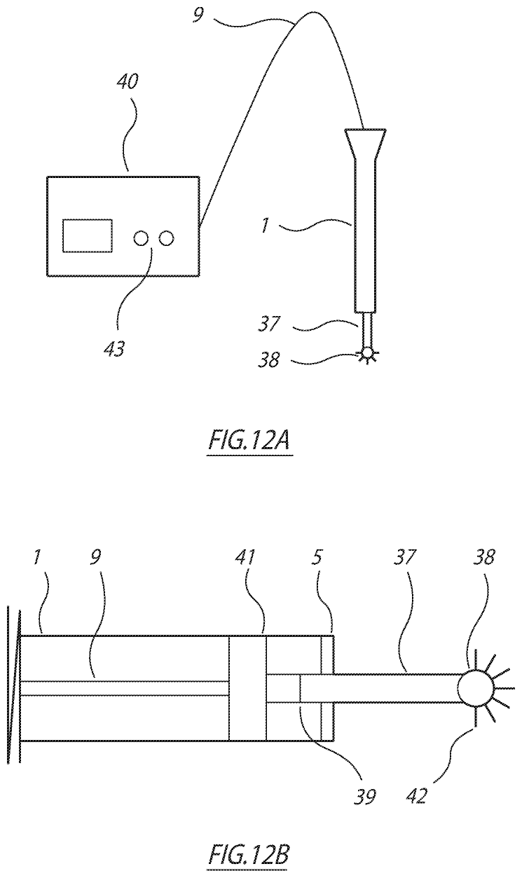

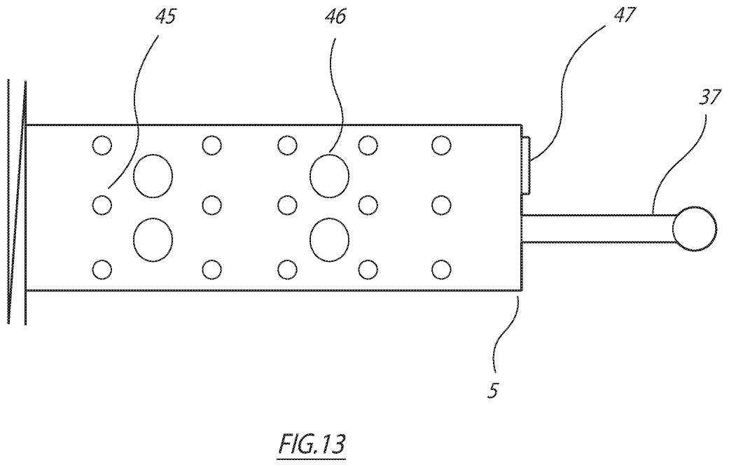

FIG. 12a schematically illustrates an exemplary waveguide (37) and its tip (38), that is powered by an external vibration control unit (40), however the waveguide and a vibrating motor (not shown) are located inside the endoscope (1). The control unit's (40) power output, duty cycle and other functions are controlled via the unit's controls (43). The control unit is connected to and powers the vibrating motor located in the endoscope via a cable (9) that runs inside the endoscope (1).

FIG. 12b schematically illustrates a close up of the distal part of the exemplary endoscope (1), showing where the waveguide (37) with a rounded tip (38) connected via an attachment mechanism (39) to the vibrating motor (41), which is connected to the external control unit via a cable (9), extends past the endoscope tip (5) in order to vibrate in an oscillating motion and transfer its energy and acoustic waves to the surrounding liquid which transmits the acoustic waves to the tissue to be treated. The waveguide has a rounded tip (38) attached to its distal end in order to enhance and increase the transmission of the acoustic energy to the surrounding liquid. The tip has protrusions (42) extending from its surface, in this example in the shape of bristles, which further enhance the mixing of the liquid medium between the tip (38) and the tissue being treated to cause stable, inertial cavitation and acoustic streaming. The endoscope can be maneuvered close to the tissue that is being treated as to maximise the amount of energy output of the waveguide reaching the tissue being treated.

FIG. 13 schematically illustrates the distal part of an exemplary colonoscope with a confocal microscope array (47) built-into the colonoscope tip (5). An ultrasonic waveguide (37) is extended via the colonoscope's working channel to transmit the ultrasonic energy to the surrounding tissue. Spray holes (45) and larger aspiration openings (46) are situated proximally to the colonoscope tip and extend back wards in order to clean the instrument and lubricate the surrounding mucosa during treatment. Another purpose of these spray and aspiration holes is to enhance the mixing of the anti-biofilm liquid in the area being treated to improve its effect and to remove any debris that may be present.

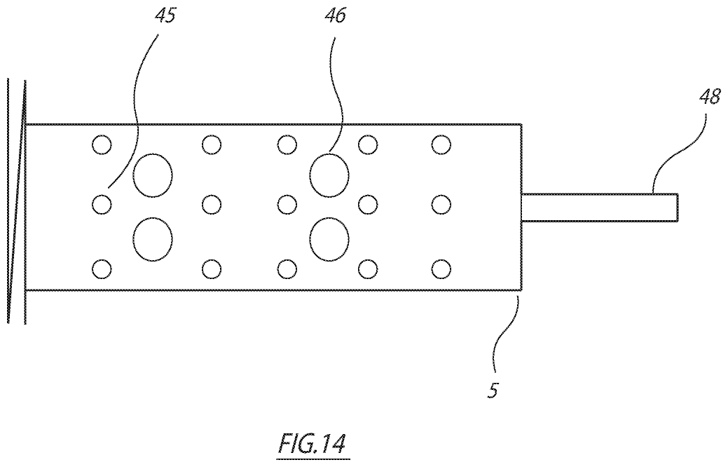

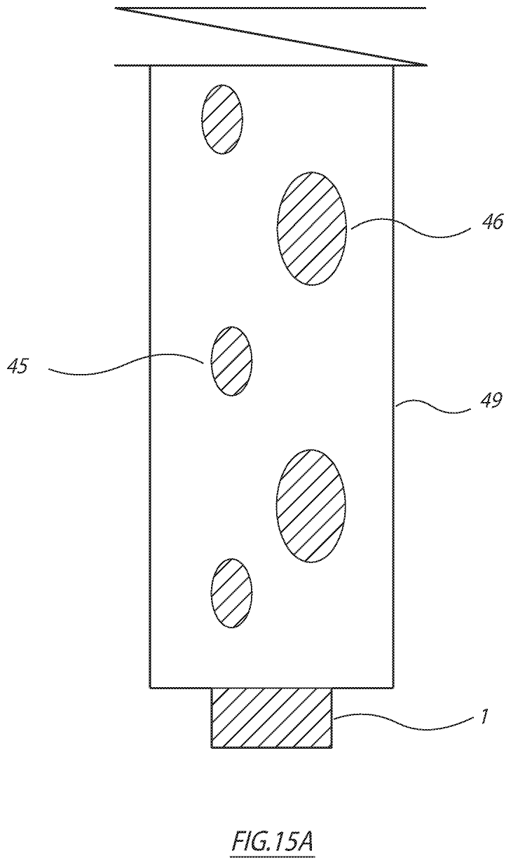

FIG. 14 schematically illustrates the distal part of an exemplary colonoscope with a confocal microscope probe (48) extended via the exemplary colonoscope's working channel to inspect for the presence or removal of biofilm from the surrounding tissue. Spray holes (45) and larger aspiration openings (46) are situated proximally to the colonoscope tip (5) and extend back wards in order to clean the instrument and lubricate the surrounding mucosa during treatment. Another purpose of these spray and aspiration holes is to enhance the mixing of the anti-biofilm liquid in the area being treated to improve its effect and to remove any debris that may be present.

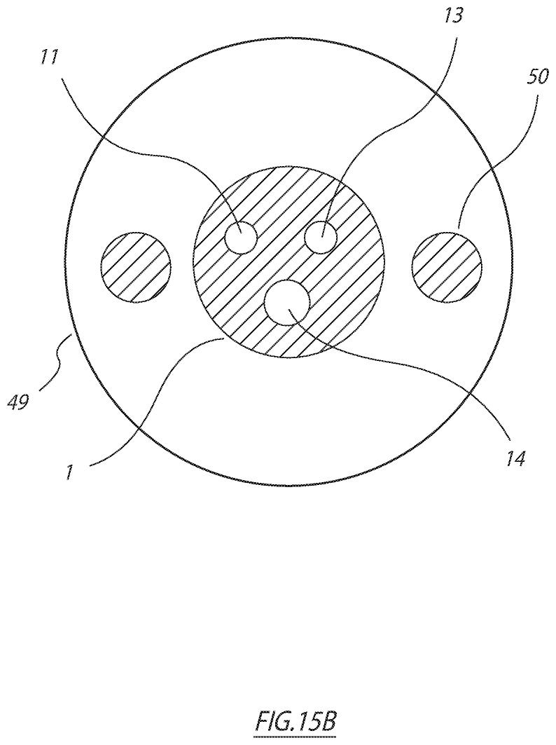

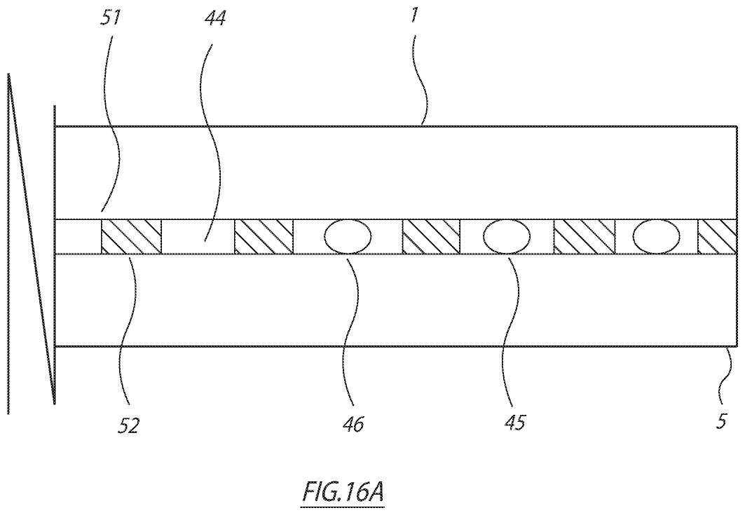

FIG. 15a schematically illustrates the lateral view of the distal part of the exemplary colonoscope (1) with a overtube (49) inserted over the colonoscope. The overtube has spray hole/s (45) along its longitudinal axis which serve to introduce liquid/s, for example, biofilm dispersion and microbiome modulating compositions into the colon. The overtube and its components (tubes, channels etc) are connected to an external unit situated proximally to the overtube (not shown) that pumps and aspirates the liquid and other compositions, for example, microbiome modulating compositions, introduced into the colon and controls the overtube's functions like the pressure and temperature of the introduced liquids. The unit may be operated with controls on the unit and/or a foot pedal situated near the physician carrying out the procedure. The spray hole/s release the liquid/s or introduced composition/s in a lateral or substantially lateral projection. The overtube also has aspirating opening/s (46) which serve to rapidly empty the colon of the introduced liquid/s in order to allow for the introduction of microbiome modulating composition/s into the colon to assist the engraftment of the newly introduced microbiota and the establishment of a non-pathogenic biofilm. An additional (optional) purpose of these spray and aspiration openings is to enhance the mixing of the anti-biofilm liquid in the area being treated via the concurrent introduction and aspiration of the liquid to improve its effect and to remove any debris that may be present.

FIG. 15b schematically illustrates the frontal view of the distal part of the exemplary colonoscope (1) with a overtube (49) inserted over the colonoscope as per diagram 15a. The colonoscope's camera (13), irrigation/water jet (11) and working channel (14) can be seen. The overtube from this view may have one or more working channels (50) built into it to introduce additional tools into the area being treated, for example, a baloon, or to remove a tissue from the colon, for example, a polyp, in case the colonoscope's working channel is being used for another purpose. These working channel/s in some embodiments may be used to introduce and aspirate liquid/s, for example, biofilm dispersion and microbiome modulating compositions into the colon when the overtube does not have lateral facing spray and aspiration openings as in diagram 15a. The overtube and its components (tubes, channels etc) are connected to an external unit situated proximally to the overtube (not shown) that pumps and aspirates the liquid and other compositions, for example, microbiome modulating compositions, introduced into the colon and controls the overtube's functions like the pressure and temperature of the introduced liquids. The unit may be operated with controls on the unit and/or a foot pedal situated near the physician carrying out the procedure. The working channel (50) may be operated as an aspirating opening which serves to rapidly empty the colon of the introduced liquid/s in order to allow for the introduction of microbiome modulating composition/s into the colon to assist the engraftment of the newly introduced microbiota and the establishment of a non-pathogenic biofilm. An additional purpose of the working channels operating as spray and aspiration openings is to enhance the mixing of the anti-biofilm liquid in the area being treated via the concurrent introduction and aspiration of the liquid to improve its effect and to remove any debris that may be present.

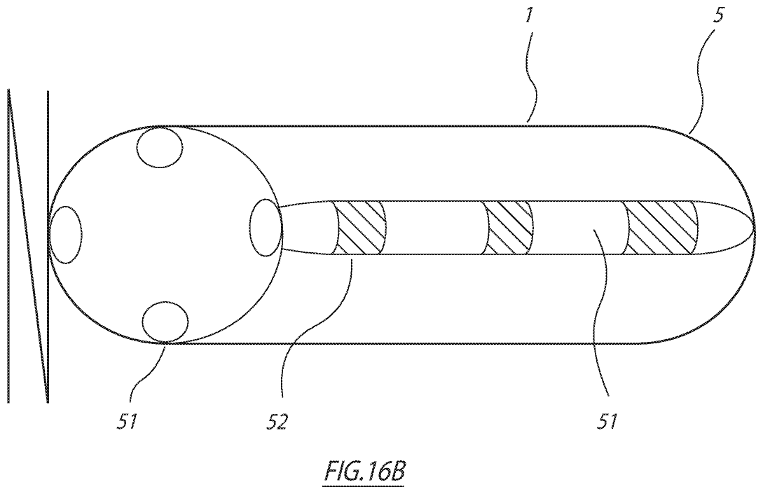

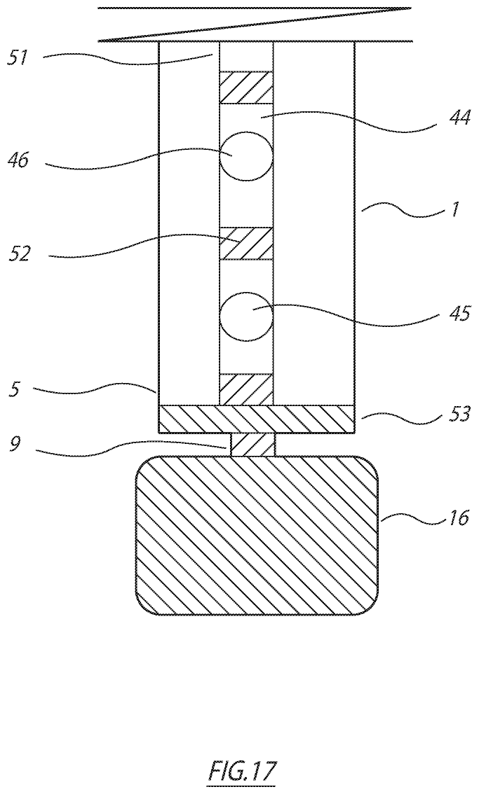

FIG. 16a shows the distal part of a colonoscope (1) with a catheter (44) running along via the endoscope's surface channel (51) to the endoscope's tip (5), the catheter (44) being kept in place by surface channel covers (52). In this embodiment a single use, multiple use or permanent catheter (44) has spray hole openings (45) and/or aspiration openings (46) at its distal end near the endoscope tip (5) to release and aspirate liquid that includes antibacterial and antibiofilm components to assist in the removal of biofilm from the surrounding tissue. The catheter/s are connected to a unit situated proximally to the endoscope and external to the patient's body which controls the infusion, aspiration, temperature, pressure etc of the liquid medium. The spray holes (45) are situated proximally to the colonoscope tip (5) and extend back wards in order to also clean the instrument and lubricate the surrounding mucosa during treatment. After the antibiofilm therapy is completed these spray holes are used to rapidly infuse the body cavity with the new microbiota in order to implant a new healthy microbiome.

FIG. 16b shows the distal part of a colonoscope (1) in a 3 dimensional drawing to display the surface channels of the endoscope (51) running along the circumference of the endoscope to its tip (5), where a single use, multiple use or permanent catheter (not shown) with spray and aspiration openings as shown in diagram 16a are being kept in place by surface channel covers (52).

FIG. 17 shows an ultrasonic emitter (16) that is wider than the diameter of the endoscope (1) that is connected to a control unit situated proximally to the endoscope and external to the patient's body. The ultrasonic emitter (16) in this embodiment may transmit ultrasound in a radial or omnidirectional mode and is powered and kept in place by a cable electrode (9) that fits in the endoscope's working channel that is made of a material as to prevent the ultrasonic emitter from moving too much once it is inserted in the body cavity being treated. Due to its size the ultrasound emitter (16) is attached to the endoscope before it is inserted in the body. The cable (9) is inserted via the endoscope's distal tip (5) and once it has exited the proximal end of the endoscope it is connected to the ultrasound emitter's control unit. In this embodiment there is a light emitter (53) placed close to the endoscope tip (5) which emits light in the 200 nm to 1000 nm range to enhance the antibacterial effect of the liquid infused in the area being treated with or without the addition of biofilm revealing stains. In this embodiment the endoscope has a surface channel (51) as shown in diagrams 16a and 16b running the length of the endoscope (1) to its tip (5) and a single use, multiple use or permanent catheter (44) with both spray holes (45) and aspiration holes (46) which can be used interchangeably and controlled by a unit situated proximally to the endoscope and outside of the patient's body. The catheter (44) is kept in place by surface channel covers (52) which keep the catheter tightly in the channel so it does not protrude from the surface of the endoscope (1).

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

A number of embodiments of the invention have been described. Nevertheless, it can be understood that various modifications may be made without departing from the spirit and scope of the invention. Accordingly, other embodiments are within the scope of the following claims.

1. A product of manufacture for debriding or disrupting a biofilm in situ, comprising an endoscope having an outer body or sheath and an inner lumen, wherein the endoscope comprises one or a plurality of ultrasound emitters, and/or one or a plurality of ultrasound ring-shaped transducers,
and the one or a plurality of ultrasound emitters, or the one or a plurality of ring-shaped ultrasound transducers, each generate an ultrasound wave that can travel perpendicularly and/or radially away from the longitudinal axis of the endoscope.

2. The product of manufacture of claim 1,
wherein the one or a plurality of ultrasound emitters are flexible and are wrapped around the outer body of the endoscope, or the one or a plurality of ultrasound emitters are placed or positioned on the outer body of the endoscope, and optionally the one or a plurality of ultrasound emitters are flat against the outer body or sheath or do not substantially protrude from or minimally protrudes from the outer body or sheath.

3. The product of manufacture of claim 1,
wherein the one or a plurality of ultrasound emitters are placed or positioned within or attached to the inside of the outer body or sheath, and the outer body or sheath substantially comprises a material which does not attenuate or alter the frequency of an ultrasound wave emitted by the one or a plurality of ultrasound emitters.

4. The product of manufacture of claim 1,
wherein the one or a plurality of ultrasound emitters comprise one or a plurality of disk shaped ultrasound emitters at regular intervals horizontally and longitudinally along the endoscope body,
and optionally the one or a plurality of ultrasound are placed as illustrated in FIG. 8.

5. The product of manufacture of claim 1,
wherein the one or a plurality of ultrasound emitters, or the one or the plurality of ring-shaped ultrasound transducers, are placed or positioned along the distal half of the length of the endoscope, or are placed or positioned along the along the distal third or quarter of the length of the endoscope.

6. The product of manufacture of claim 1,
wherein the one or a plurality of ultrasound emitters, or the one or the plurality of ring-shaped ultrasound transducers, transmit ultrasound energy in a continuous or pulsed mode.

7. The product of manufacture of claim 1, further comprising an electrode or electric cable running thorough the inner lumen, and the electrode cable is operatively connected to the one or a plurality of ultrasound emitters, or the one or plurality of ring-shaped ultrasound transducers, to power the one or a plurality of ultrasound emitters or the one or plurality of ring-shaped ultrasound transducers.

8. The product of manufacture of claim 1, further comprising one or a plurality of temperature sensors.

9. The product of manufacture of claim 1, wherein the one or plurality of ring-shaped transducers are placed at regular intervals inside the entire length, or along a section of, the endoscope's sheath or outer body,
wherein optionally the ring-shaped transducers are placed every 3 to 10 or 20 cm, or every 10 to 30 cm, along the length of and inside the endoscope's sheath or outer body, and optionally the ring-shaped transducers are placed or positioned along the distal half of the length of the endoscope, or are placed or positioned along the along the distal third or quarter of the length of the endoscope.

10. The product of manufacture of claim 1, further comprising a wide beam ultrasound emitter array,
wherein optionally the wide beam ultrasound emitter array is a built-in component of the endoscope or is an attachment to the endoscope, and optionally the wide beam ultrasound emitter array is a removable attachment to the endoscope,
and optionally the wide beam ultrasound emitter array transmits ultrasound waves perpendicularly and/or radially away from the longitudinal axis of the endoscope,
and optionally the wide beam ultrasound emitter array comprises a device as set forth in FIG. 5 or FIG. 6,
and optionally the wide beam ultrasound emitter array is positioned no closer to between about 5 cm to 20 cm from the distal end or tip of the endoscope,
and optionally the electrode or electric cable running thorough the inner lumen is operatively connected to the wide beam ultrasound emitter array and powers the wide beam ultrasound emitter array,
and optionally the wide beam ultrasound emitter array transmits ultrasound energy in a continuous or pulsed mode,
and optionally the wide beam ultrasound emitter array has a curvilinear shape, and optionally the wide beam ultrasound emitter array comprises a lifting mechanism which can lift the wide beam ultrasound emitter array to between 1 to 90 degrees from the longitudinal axis of the endoscope, and optionally the lifting mechanism is operatively connected to a control mechanism capable of activating the lifting mechanism to lift the wide beam ultrasound emitter array to between 1 to 90 degrees from the longitudinal axis of the endoscope, or to close the wide beam ultrasound emitter array back against the body of the endoscope.

11. The product of manufacture of claim 1, wherein the product of manufacture further comprises a plurality of spacing rings projecting out from the body of the product of manufacture at regular intervals to prevent the product of manufacture from resting against a tissue when the product of manufacture is inserted into the body, optionally inserted into a colon,
and optionally the plurality of spacing rings project out from the body of the product of manufacture between about 2 to 20 cm,
and optionally the plurality of spacing rings are spaced between about 3 to 30 cm along the length of the product of manufacture,
and optionally the plurality of spacing rings comprise flexible spacing rings.

12. The product of manufacture of claim 1, wherein the product of manufacture further comprising an attachment that is secured to the distal end of the product of manufacture, wherein the attachment comprises an array of radial ultrasound emitters, optionally ring ultrasound transducers, situated or placed intermittently along the length of the attachment, and the attachment is operably connected to the electrode or electric cable running thorough the inner lumen of the product of manufacture to power the radial ultrasound emitters,
and the optionally the attachment further comprises a plurality of spacing rings situated or placed intermittently along the length of the attachment,
and optionally the attachment has a rounded tip or end, and optionally the rounded tip or end comprises a plurality of bristles or equivalent protrusions,
and optionally the attachment is configured as set forth in FIG. 9.

13. The product of manufacture of claim 1, wherein the product of manufacture further comprises a vibrating motor operatively connected to an external control unit via a cable, and the vibrating motor is operatively connected to a waveguide with a rounded tip or end that can be extended past or outside of the distal end of the product of manufacture, and the vibrating motor when activated causes the rounded tip or end to vibrate in an oscillating motion,
and optionally the rounded tip or end comprises a plurality of bristles or equivalent protrusions,
and optionally the vibrating motor, waveguide and rounded tip or end are configured as set forth in FIG. 12a or FIG. 12b.

14. The product of manufacture of claim 1, wherein the product of manufacture further comprises a microscope array, optionally a confocal microscope array, built-into the end or tip of the product of manufacture.

15. The product of manufacture of claim 1, wherein the product of manufacture further comprises:
(a) a plurality of liquid spray holes; (b) a plurality of aspiration openings; or (c) a combination of (a) and (b),
and the plurality of aspiration openings are operatively connected to a plurality of tubes to allow aspiration of fluids or liquids from a tissue space surrounding the product of manufacture when the product of manufacture is inserted in a body space in situ,
and the plurality of liquid spray holes are operatively connected to a plurality of tubes to allow spraying or ejection of fluids or liquids under pressure out from the product of manufacture into a tissue space surrounding the product of manufacture when the product of manufacture is inserted in a body space in situ, and optionally the plurality of liquid spray holes are extended, angled or pointed back away from the distal end of the product of manufacture, optionally configured to allow a liquid or a fluid sprayed from the plurality of liquid spray holes to cleanse the product of manufacture,
wherein optionally plurality of aspiration openings have a larger diameter than the plurality of liquid spray holes,
and optionally the plurality of aspiration openings and/or the plurality of liquid spray holes are configured as set forth in FIG. 13 or FIG. 14,
and the plurality of aspiration openings and/or the plurality of liquid spray holes are situated in the distal half, third or quarter end of the product of manufacture.

16. The product of manufacture of claim 1, further comprising an overtube fitted along the outer circumference of the product of manufacture,
wherein optionally the overtube comprises plurality of liquid spray holes operatively connected to a plurality of tubes to allow spraying or ejection of fluids or liquids under pressure out from the product of manufacture into a tissue space surrounding the product of manufacture when the product of manufacture is inserted in a body space in situ, and optionally the plurality of liquid spray holes are extended, angled or pointed back away from the distal end of the product of manufacture, optionally configured to allow a liquid or a fluid sprayed from the plurality of liquid spray holes to cleanse the product of manufacture,
and optionally the overtube comprises plurality of aspiration openings operatively connected to a plurality of tubes to allow aspiration of fluids or liquids from a tissue space surrounding the product of manufacture when the product of manufacture is inserted in a body space in situ,
and optionally the overtube comprises a one or a plurality channels having lumens capable of having inserted therein a tube or an instrument, and optionally an instrument inserted into one or in a plurality channels is capable of delivering and inflating a balloon into a body space in situ, and optionally an instrument inserted into one or in a plurality channels is capable of delivering a therapeutic solution or formulation, and optionally the therapeutic solution or formulation comprises a biofilm dissolving or disrupting agent, a soap, an antibiotic or a fecal microbiota transplantation formulation,
and optionally the plurality of aspiration openings and/or the plurality of liquid spray holes are configured as set forth in FIG. 15a or FIG. 15b.

17. The product of manufacture of claim 1, wherein the product of manufacture is configured or manufactured as a product of manufacture, device or endoscope as set forth in FIG. 1, FIG. 2, FIG. 3, FIG. 4, FIG. 7, FIG. 9, FIG. 10A, FIG. 10B, FIG. 11A, FIG. 11B, FIG. 12A, FIG. 12B, FIG. 13, FIG. 14, FIG. 15A or FIG. 15B.

18. A method for debriding or disrupting a biofilm in situ, comprising use of a product of manufacture of claim 1.

19. A for debriding or disrupting a biofilm in situ comprising a product of manufacture of claim 1.

20. The product of manufacture of claim 8, wherein:
the one or a plurality of temperature sensors are spaced along the length of the endoscope,
the one or a plurality of temperature sensors are operatively connected to a display or a control panel that displays a temperature reading by the one or a plurality of temperature sensors to an operator, or
the one or a plurality of temperature sensors are operatively connected to a computer capable of monitoring the temperature and turning off the power to the one or a plurality of ultrasound emitters when the temperature reaches a predetermined temperature setting.
